SEQUENCE LISTING (9 sequences)

SEQ ID NO: 1 - DNA, 1246 bp, Rattus sp.
ggcagccgcgaagtcacttggttgctctcctcaagtccatggatgtgcatctgtttgactatgtgggaacctggaactactcggacatcaactggccctgtaacagtagcgactgcatcgtcgtggacaccgtgcagtgtcccgccatgcccaacaagaatgtgctgctgtataccctctccttcatctacattttcatcttcgtgatcggtatgattgccaactccgtggtggtctgggtgaatatccaggccaagactacaggctacgacacacactgctacatcttgaacctggccattgccgatctgtgggtcgtcatcaccatccctgtctgggtggtcagtctcgtgcagcataaccagtggcccatgggtgagctcacgtgcaagatcacacacctcattttctccatcaacctctttgggagcatcttcttcctcgcatgcatgagcgtggaccgctatctctccatcacctacttcaccagcacctccagctataagaagaagatggtacgccgtgttgtctgcgtcttggtgtggctgctggccttctttgtgtccctgcctgacacctactacctgaagacggtcacatctgcttccaacaacgagacctactgcaggtccttctaccccgagcacagcatcaaggagtggctcattggcatggagctggtctccgtcatcttgggttttgctgtccccttcaccatcattgctatcttctacttcctgctcgccagagccatgtcagcatccggtgaccaggagaaacacagcagccggaagatcatcttctcctacgtggtggtcttcctggtgtgttggctgccgtaccattttgtggttctgctggacatcttctctatcttgcactacatcccgttcacctgccaactcgagaatgtgctctttacagcgctgcacgtcacgcagtgcctgtccctggtgcactgctgtgtcaaccctgtgctctacagcttcatcaaccgaaactacaggtacgagctgatgaaggccttcatcttcaagtactcagccaaaacaggactcaccaaactcatcgatgcctccagagtgtcagagacagagtactctgccctggagcagaacaccaagtgaccgtgctataggagcatgggggacatgtgcatgttgcaaatggggcagctgggccctgcggtttcttcaagaaaagcactgtagctttgggtctggttgcttgagtggtatgaagaggg

SEQ ID NO: 2 - DNA, 1086 bp, Homo sapiens
atggatctgcacctcttcgactacgccgagccaggcaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcagcggcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtgtcggagacggagtactccgccttggagcaaaacgccaag

SEQ ID NO: 3 - protein, 362 aa, Rattus sp.
MDVHLFDYVEPGNYSDINWPCNSSDCIVVDTVQCPAMPNKNVLLYTLSFIYIFIFVIGMIANSVVVWVNIQAKTTGYDTHCYILNLAIADLWVVITIPVWVVSLVQHNQWPMGELTCKITHLIFSINLFGSIFFLACMSVDRYLSITYFTSTSSYKKKMVRRVVCVLVWLLAFFVSLPDTYYLKTVTSASNNETYCRSFYPEHSIKEWLIGMELVSVILGFAVPFTIIAIFYFLLARAMSASGDQEKHSSRKIIFSYVVVFLVCWLPYHFVVLLDIFSILHYIPFTCQLENVLFTALHVTQCLSLVHCCVNPVLYSFINRNYRYELMKAFIFKYSAKTGLTKLIDASRVSETEYSALEQNTK

SEQ ID NO: 4 - protein, 362 aa, Homo sapiens
MDLHLFDYAEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFSGIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQNAK

SEQ ID NO: 5 - protein, 10 aa, Artificial
  example conservatively substituted variation of amino acids 1-10 of SEQ ID NO4
MDIHLYDYAE

SEQ ID NO: 6 - protein, 10 aa, Artificial
  example conservatively substituted variation of amino acids 1-10 of SEQ ID NO4
MDLHLFEWAE

SEQ ID NO: 7 - DNA, 1150 bp, Rattus sp.
  misc_feature (11)..(11): unknown
aattccttttnccgcacatgacaggtttattgacaaaggctgaacgtcaccagcctcatcagcttttttnacatttnatgggaaggtttttacatcatcaccgcagttgtccccagctgttatttctgtagaaacaaattcgtcagccacaggtgctggagtatttcattgtcctgtgcaccttcatgtcgctcttcctgcaggtcaacatgtacagcagcgtcttcttcctcacctggatgagcttcgaccgtacatcgccctggccagggccatgcgctgcagcctgttccgnaccaaacaccacgcccggctgagctgtggnctcatctggatggcatccgtgtcagccacgctggtgcccttcaccgccgtgcacctgcancacaccgacgangcctgcttctgtttcgcggatgtccgggaggtgcantggctcgaggtcacgctgggcttcatcgtgcccttcgccatcatcggcctgtgctactccctcattgtccgggtgctggtcagggcgcaccggcaccgtgggctgcggccccggcggcagaangcgctccgcatgatcctcgcggtggtgctggtcttcttcttctgctggctgccggaaaacntcttcatcancgtgcacctcctgcancggacgcaacctggggccgctccctgcaagcagttttttccgccatgcccaccccctcacgggccacattgtnaacntcgccgcttntccaacagctgcctaaaccccttcatntacagctttctcggggagaccttcagggacaagctgaggctgtacattgagcagaaaacaaatttgccggccctgaaccgcttctgtcacgctgccctgaaggccgtcattccagacagcaccgagcagtcggatgtgaggttcagcagtgccgtgtagacagccttggccgcataggcccagccagggtgtgactcgggagctgcacacacctgggtggacacaaggcacggccacgtcatgtctctaaactgcggttagatgtggcttctggctcctcggggcctngcgagggtnaagcttgcctggtcancctggggctgcttaggaaacctnacgactggtcaccttgcactcctcacanagaattgctacaatnccaaagggctcgcc

SEQ ID NO: 8 - DNA, 950 bp, Rattus sp.
aacatggccgtcgcggacctgggcatcatcctgtctctgcctgtgtggatgctggaggtcatgctggtctacacctggctctggggcagcttctcctgtcgcttcattcattatttctaccttgccaacatgtacagcagcatcttcttcctcacctgcctcagcattgaccgctacgtcaccctcaccaatacctctccctcctggcagcgccaccagcaccgaatacggagggccgtgtgcgcaggcgtctgggtcctctccgccatcatcccactgcctgaggtggtacatatccagctgctggatggctccgagcccatgtgcctcttcctagcaccttttgaaacgtacagcgcgtgggccctggcagtggccctgtcggctaccatcctgggcttcctactgccttttcctctcatcgcagtgtttaatatcctgtcagcctgccggcttcggaggcaagggcagacagagagcaggcgccactgtctgttgatgtccgcttacatagttgtctttgtcatctgctggctgccctaccacgtgactatgctgctgctcactctgcacacaacccacatcttccttcactgcaacctggttaacttcctctacttcttctacgaaatcattgactgcttctccatgctacactgtgtcgccaaccccatcctctacaactttctcagcccgagcttccggggccgactgctgagccttgtggtccgttaccttcccaaggagcaggccgcagcaggcggtcgagcctcctcttcatgttccacccagcactccatcatcattaccaaagagggcagcctgccgctgcagcggatctgcacacccccgccatcagaaacgtgcaggcctcctctctgcctccgaacacctcacctacactctgcaattccatagccagctaaggtagattctagcttcttccacca

SEQ ID NO: 9 - DNA, 1089 bp, Canis familiaris
atggatctgcacctcttcgactacgccgagccaggcaacttctccgacataagctggccgtgcaacagcagcgactgcatcgtcgtggacaccgtgctgtgccccaacatgcccaacaaaagcgtgctgctgtacacgctgtccttcatttacatcttcatcttcgtgatcggcatgatcgccaactccgtggtggtctgggtgaacatccaggccaagaccaccggctacgacactcactgctacatcctcaacctggccatcgccgacctgtgggtggtcgtcaccatccccgtctgggtggtcagcctcgtgcagcataaccagtggcccatgggggagctcacgtgcaagatcacgcacctcatcttctccatcaacctgttcggcagcatcttcttcctcacgtgcatgagcgtggaccgctacctctccatcacctacttcgccagcacgtcgagccgcaggaagaaggtggttcgccgcgccgtctgtgtcctggtgtggctgctggccttctgcgtgtccctgcccgacacctactacctgaagaccgtcacgtcggcgtccaacaacgagacctactgccgctccttctaccccgagcacagcgtcaaggagtggctcatcagcatggagctggtctcggtggtcctgggcttcgccatccccttctgcgtcatcgccgtcttctactgcctgctggcccgcgccatctccgcgtccagcgaccaggagaagcagagcagccgaaagatcatcttctcctacgtggtggtcttcctcgtgtgctggctcccctaccacgtggtggtgctcctggacatcttctccatccttcactacatccccttcacctgccagctggagaacttcctcttcacggctctgcacgtcacgcagtgcctgtctctggtgcactgctgcgtcaaccccgtgctctatagcttcatcaaccgtaactacagatacgagctgatgaaggccttcatctttaagtactcggccaagacgggtctcaccaagctcatcgatgcctccagggtgtcggagacggagtactccgccttggagcaaaacgccaagtga

(19) United States
(12) Patent Application Publication
(10) Pub. No.: US 2004/0110170 A1
(43) Pub. Date: Jun. 10, 2004

(54) Cloning and characterization of calcitonin gene related peptide receptors

(75) Inventors: Joseph R. Pisegna, Santa Monica, CA (US); Stephen A. Wank, Potomac, MD (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/440,479

(22) Filed: May 16, 2003

Related U.S. Application Data
(60) Provisional application No. 60/381,911, filed on May 18, 2002.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

This invention provides CGRP receptors (including both amino acid and nucleic acid sequences). Compositions which include these polypeptides, proteins, nucleic acids, recombinant cells, transgenic animals, and antibodies to the receptors are also provided.

### CROSS-REFERENCE TO RELATED APPLICATIONS

This application claims the benefit of U.S. Provisional Application No. 60/381,911 filed May 18, 2002, entitled “CLONING AND CHARACTERIZATION OF CALCITONIN GENE RELATED PEPTIDE RECEPTORS” and naming Joseph Pisegna et al. as the inventors. This prior application is hereby incorporated by reference in its entirety.

### FIELD OF THE INVENTION

The invention relates to the field of neuropeptide receptors. In particular, the invention relates to amino acid and nucleic acid sequences for CGRP receptors and methods for producing and isolating such sequences or molecules. The invention also provides methods for use of such molecules (e.g., for identifying, isolating, and/or purifying agonists/antagonists of CGRP receptors), compositions comprising the molecules, and homologous molecules, as well as antibodies to the molecules.

### BACKGROUND OF THE INVENTION

The Calcitonin Gene Related Peptide (CGRP) is a 37-amino acid neuropeptide encoded as a splice variant by the calcitonin/CGRP gene. See, e.g., S. G. Amara, et al. (1982) Nature 298:240-244 and M. G. Rosenfeld, et al. (1983) Nature 204:129-135. CGRP peptides are structurally similar to members of the Adrenomedullin and Amylin families (e.g., including similar N-terminal disulfide bonds and C-terminal amide). Two biologically active forms of CGRP (α-CGRP and β-CGRP) exist in both rats and humans and induce similar biological activities. CGRP receptors are highly expressed throughout the brain and gastrointestinal tract where they are involved in, e.g., regulation of pain responses and gut motility. CGRP has also been found to be involved in numerous important physiological activities, such as: vasodilatation, cardiac acceleration, inhibition of gastric acid secretion, reduction of intestinal motility, regulation of glucose metabolism, diminution of appetite, and reduction of growth hormone release, etc. See, e.g., S. J. Wimalawansa, (1997) Crit Rev in Neurobiol 11, 167-239.

CGRP has been well conserved throughout evolution and is widely distributed throughout several tissues including nerve, cardiovascular, gastrointestinal, endothelial, and smooth muscle. Additionally, CGRP is abundant in sensory afferent nerves and central nervous system (CNS) neurons, suggesting that it plays an important role in mediating visceral afferent sensation. In the gastrointestinal (GI) tract, CGRP-immunoreactive nerve fibers have been identified in the stomach, duodenum, and in the small and large intestine.

Prior to the cloning of any gene for a CGRP receptor (CGRP-R), two classes of receptors had been defined through pharmacological studies using brain membrane preparations and tumor cell lines that express receptors for CGRP. See, e.g., D. T. Fournier et al. (1990) J Pharmacol Exp Ther 254:123-8 and D. T. Fournier et al. (1989) J Pharmacol Exp Ther 251:718-25. In these studies, tissues were exposed to both the selective antagonist, CGRP (8-37), and the selective agonist, [Cys(ACM)2,7]hCGRP. The results suggested the presence of at least two CGRP-R subtypes based upon the different pharmacological profiles of the receptors studied. For example, CGRP (8-37) displayed relatively potent, competitive antagonist properties toward the action of native CGRP in guinea pig atrial and ileal preparations and on the central nervous system's mediated inhibition of CGRP food intake, while, in the rat vas deferens, the antagonistic potency of CGRP (8-37) was much reduced. This early work suggested the existence of several distinct classes of CGRP receptor subtypes.

Furthermore, it has also been determined that a distinct receptor with seven transmembrane domains, the calcitonin-receptor-like receptor (CRLR), can function as either a CGRP receptor or Adrenomedullin receptor. See, e.g., L. M. McLatchie et al., (1998) Nature 393:333-339. The CRLR's pharmacological identity depends upon the co-expression of a family of single transmembrane domain proteins called RAMPs (receptor associated modifying proteins), which function to transport the CRLRs to the plasma membrane. For example, when the RAMP protein variant RAMP1 is co-expressed, the CRLR has CGRP receptor pharmacology.

Recently, distinct from the CRLR receptor, a CGRP receptor has been cloned as an orphan CGRP receptor in dogs (RDC-1), see, S. Kapas, et al., (1995) Biochem Biophys Res Commun 217:832-8. Additionally, a chaperone protein required for CGRP receptor expression has been described in humans. See, S. M. Foord, et al., (1987) Eur J Biochem 170:373-9.

A welcome addition to the art would be the identification and isolation of sequences and characterization of molecules of additional CGRP receptors. The present invention provides these and other benefits which will be apparent upon examination of the following specification and figures.

### SUMMARY OF THE INVENTION

The invention provides CGRP-receptors (e.g., receptors which bind CGRP molecules and/or CGRP-like molecules optionally wherein such binding elicits changes in (optionally intracellular) cAMP concentration, production, etc., and optionally wherein the CGRP molecules, etc., are bound by CGRP-receptor), as well as polypeptides, compositions, nucleic acids, antibodies to the receptors, and vectors comprising the receptor sequences, etc.

In some aspects, the current invention comprises an isolated or recombinant expression vector comprising a nucleic acid which comprises a sequence encoding a CGRP receptor. Such sequence is optionally selected from: a polynucleotide sequence of SEQ ID NO:1 or SEQ ID NO:2 (or complementary sequences thereof); a polynucleotide sequence encoding the polypeptide sequence of SEQ ID NO:3 or SEQ ID NO:4 (or complementary sequences thereof); a polynucleotide sequence that hybridizes under highly stringent conditions over substantially the entire length of the above polynucleotides (and optionally which is unique as compared to a sequence corresponding to GenBank accession number X14048); and a polynucleotide sequence comprising a fragment of any of the above wherein the fragment encodes a CGRP receptor (e.g., wherein it binds CGRP or a CGRP like molecule and optionally wherein such binding elicits a cAMP change, e.g., intracellularly). Additionally, the invention comprises an isolated or recombinant nucleic acid which encodes a CGRP receptor (optionally a rat CGRP-receptor or a human CGRP-receptor) and where the polypeptide encoded by such nucleic acid is substantially identical over at least about 200, at least about 225, at least about 250, at least about 275, at least about 300, at least about 325, at least about 350, at least about 355, at least about 360, at least about 361, or at least about 362 contiguous amino acid residues of SEQ ID NO:3 (or SEQ ID NO:4). Optionally, the invention includes any of the above nucleic acids wherein the encoded polypeptide comprises one or more leader sequence, epitope tag sequence, carboxy terminal epitope tag sequence, or GFP sequence (or other sequence used for, e.g., identification, tracking, targeting, etc. of the protein) or wherein the polypeptide comprises a fusion protein comprising one or more additional sequences. The invention also comprises compositions of matter comprising two or more of any of the above nucleic acids (and optionally wherein such compositions comprise a library of at least about 2, at least about 5, at least about 10, at least about 15, at least about 20, at least about 25, at least about 50, at least about 75, or at least about 100, or more nucleic acids). Other compositions of the invention include those produced through cleavage of one or more of the above expression vector nucleic acids. In some embodiments the cleavage is, e.g., through mechanical cleavage, chemical cleavage, enzymatic cleavage, cleavage with a restriction endonuclease, cleavage with a RNAse, or cleavage with a DNAse. Yet other compositions of the invention include those produced through a process of incubating one or more of the above nucleic acids with deoxyribonucleotide triphosphates and a nucleic acid polymerase (optionally a thermostable polymerase). Other embodiments of the invention include cells comprising one or more nucleic acid described above. Other cells optionally express a polypeptide encoded by such nucleic acids (optionally wherein the polypeptide comprises a receptor, optionally a rat receptor or optionally a human receptor, which can bind CGRP and/or CGRP-like molecules and optionally wherein such binding elicits a change in cAMP concentration, e.g., intracellularly, etc.). In some embodiments herein, the invention comprises a vector comprising any of the above nucleic acid sequences. Such vector optionally comprises a plasmid, cosmid, phage, virus, virus fragment, expression vector, etc. Cells transduced by such vectors are also included.

In other aspects, the current invention comprises an isolated or recombinant polypeptide encoded by a nucleic acid of the invention (e.g., as described above). Such isolated or recombinant polypeptide optionally comprises an amino acid sequence of SEQ ID NO:3 (or optionally of SEQ ID NO:4). The isolated or recombinant polypeptides optionally encode a receptor for a CGRP molecule or a receptor for a CGRP-like molecule. Optionally the binding of a CGRP molecule or a CGRP-like molecule to such polypeptide of the invention elicits a change in cAMP concentration (e.g., intracellularly). Compositions comprising such isolated or recombinant polypeptides are also features of the invention. Additionally, options wherein such compositions comprise CGRP and/or CGRP-like molecules bound to the polypeptides are also included.

Other aspects of the invention comprise an isolated or recombinant polypeptide (optionally comprising a CGRP receptor) comprising a sequence having at least 91%, at least 92%, at least 93%, at least 94%, at least 95%, at least 96%, at least 97%, at least 98%, or at least 99% or more sequence identity to SEQ ID NO: 3 (and/or optionally to SEQ ID NO:4). Optionally, such polypeptide binds a CGRP molecule or a CGRP-like molecule. Compositions including such polypeptides are also featured in the invention. Such compositions can comprise the polypeptides bound to CGRP or CGRP-like molecules (wherein optionally such bound molecules elicit a cAMP response or change, e.g., optionally intracellularly). In other embodiments, such isolated or recombinant polypeptides comprise one encoded by a polynucleotide sequence which hybridizes under highly stringent conditions over substantially the entire length of: a polynucleotide sequence of SEQ ID NO:1 (or a complementary sequence thereof) or SEQ ID NO:2 (or a complementary sequence thereof); or a polynucleotide sequence that encodes a polypeptide sequence of SEQ ID NO:3 (or optionally of SEQ ID NO:4).

In yet other aspects, the current invention comprises an isolated or recombinant polypeptide (which optionally binds a CGRP molecule or a CGRP-like molecule and which binding optionally elicits a cAMP response/change) which is encoded by a polynucleotide sequence selected from: (a) a polynucleotide sequence of SEQ ID NO:1 or 2; (b) a polynucleotide sequence encoding a polypeptide sequence of SEQ ID NO:3 or SEQ ID NO:4; (c) a polynucleotide sequence that hybridizes under highly stringent conditions over substantially the entire length of (a) or (b) wherein the polynucleotide sequence does not comprise a sequence corresponding to GenBank accession number X14084; or (d) a polynucleotide encoding a polypeptide having at least 91%, at least 92%, at least 93%, at least 94%, at least 95%, at least 96%, at least 97%, at least 98%, or at least 99% or more sequence identity to SEQ ID NO:3 or SEQ ID NO:4, wherein the polypeptide binds a CGRP molecule or a CGRP-like molecule. In some embodiments, the invention comprises a composition including such isolated or recombinant polypeptide bound to a CGRP molecule or to a CGRP-like molecule (optionally wherein the binding of the molecule elicits a cAMP response/change). Such polypeptides include those comprising an amino acid sequence that is substantially identical over at least 200, at least 300, at least 350, at least 360, at least 361, or at least 362 contiguous amino acid residues of SEQ ID NO:3 or SEQ ID NO:4.

In other aspects the invention comprises an isolated or recombinant polypeptide which comprises an amino acid sequence of SEQ ID NO:3 or SEQ ID NO:4 (optionally wherein such polypeptide comprises a receptor capable of binding a CGRP molecule or a CGRP-like molecule and optionally wherein such binding elicits a change in cAMP concentration/response). Additionally, such peptides (and also peptides as described above) comprise one or more of: a leader sequence, a precursor polypeptide, a secretion signal, a localization signal, an epitope tag, an E-tag, or a His epitope tag.

The current invention includes a method of producing a polypeptide through introducing a nucleic acid (e.g., optionally in an expression vector) of the invention (e.g., as described above) into a population of cells. Such nucleic acid optionally is operably linked to a regulatory sequence capable of directing expression of the encoded polypeptide in at least a subset of cells or progeny thereof, and propagation of the cells. Such method optionally further comprises isolating the polypeptide from the cells or the culture medium and optionally further wherein the culturing is done in a bulk fermentation vessel. The cells for such propagation optionally can comprise, e.g., bacterial cells, eukaryotic cells, fungal cells, yeast cells, plant cells, insect cells, and animal (e.g., mammalian) cells. If animal (e.g., mammalian) cells are utilized, the method optionally comprise, e.g., fertilized oocytes, embryonic stem cells (or pluripotent stem cells) and can optionally further comprise regenerating a transgenic animal (e.g., mammal) expressing the polypeptide (and also optionally recovering the polypeptide from the transgenic animal or from a by product of the animal). Additionally and/or alternatively, if animal (e.g., mammalian) cells are utilized, the method can further comprise wherein the polypeptide is overexpressed or wherein the polypeptide comprises a knockout polypeptide, either of which is optionally localized to a particular tissue or cell type in the transgenic animal.

The invention also includes a method of producing an isolated or recombinant polypeptide through introducing into a population of cells a recombinant expression vector comprising a nucleic acid of the invention (e.g., as described above), administering the expression vector into an animal (e.g., mammal), and isolating the polypeptide from the animal or from a by product of the animal.

In other aspects, the current invention features an isolated or recombinant polypeptide comprising a receptor capable of binding a CGRP molecule (or optionally, a CGRP-like molecule), wherein such binding optionally elicits a change in cAMP concentrations, production, response, etc. Such polypeptide is specifically bound by a polyclonal antisera raised against at least one antigen comprising SEQ ID NO:3 (or a fragment thereof) or SEQ ID NO:4 (or a fragment thereof) and which antisera is subtracted with a sequence corresponding to GenBank accession number X14084 (e.g., the polypeptide equivalent of such accession number).

The invention also features an antibody or antisera that is produced by administering an isolated or recombinant polypeptide of the invention (e.g., as described above) to a animal (e.g., mammal). Such antibody or antisera specifically binds at least one antigen comprising a polypeptide of the amino acid sequence of SEQ ID NO:3 or 4, but does not specifically bind to a peptide encoded by a polypeptide corresponding to GenBank accession number X14084.

Another feature of the invention includes an isolated or recombinant expression vector comprising a nucleic acid which comprises a unique subsequence in a nucleic acid represented by SEQ ID NO:1 (or SEQ ID NO:2), wherein the unique subsequence is unique as compared to a nucleic acid corresponding to the molecule of GenBank accession number X14084 (e.g., the nucleic acid equivalent of the accession number).

The invention also includes an isolated or recombinant polypeptide which comprises a unique subsequence in a polypeptide represented by SEQ ID NO:3 (or SEQ ID NO:4), wherein the unique subsequence is unique as compared to a polypeptide sequence corresponding to a wild-type CGRP receptor corresponding to GenBank accession number X14084 (e.g., the polypeptide version of the accession number reference). A composition comprising such isolated or recombinant polypeptide wherein the polypeptide is bound to a CGRP molecule or to a CGRP-like molecule is also included.

In other aspects, the invention comprises a method of modulating the activity of a CGRP receptor, wherein the receptor comprises a polypeptide sequence comprising at least 91%, at least 92%, at least 93%, at least 94%, at least 95%, at least 96%, at least 97%, at least 98%, or at least 99% or more sequence identity to SEQ ID NO:3 or SEQ ID NO:4. Such method optionally comprises binding a CGRP molecule, or a CGRP receptor agonist, or a CGRP receptor antagonist to the CGRP receptor. Such method also optionally comprises wherein the CGRP receptor comprises a human CGRP receptor or comprises a rat CGRP receptor.

The invention also features a method of performing screening of CGRP receptor modulating compounds (optionally high throughput screening). Such method comprises, e.g., interacting a putative CGRP-receptor modulating compound with a CGRP receptor of the invention, wherein the receptor comprises a polypeptide sequence comprising at least 91%, at least 92%, at least 93%, at least 94%, at least 95%, at least 96%, at least 97%, at least 98%, or at least 99% or more sequence identity to SEQ ID NO:3 or SEQ ID NO:4.

These and other objects and features of the invention will become more fully apparent when the following detailed description is read in conjunction with the accompanying figures.

### BRIEF DESCRIPTION OF THE DRAWINGS

FIG. 1: Displays an agarose electrophoresis gel with RT-PCR products.

FIG. 2: Displays the ability of CGRP to increase intracellular cAMP in NIH/3T3 cells stably expressing the JPr-CGRP receptor.

FIG. 3: Displays the ability of CGRP to increase intracellular cAMP in NIH/3T3 cells stably expressing the JPr-CGRP receptor.

FIG. 4: Displays the ability of CGRP to increase intracellular cAMP in NIH/3T3 cells stably expressing the Skb-CGRP receptor.

FIG. 5: Displays the ability of CGRP to increase intracellular cAMP in NIH/3T3 cells stably expressing the Skb-CGRP receptor.

FIG. 6: Displays the ability of CGRP to increase intracellular cAMP in NIH/3T3 untransfected control cells.

FIG. 7: Displays the ability of CGRP to inhibit ¹²⁵I-CGRP binding in NIH/3T3 cells stably expressing the JPr-CGRP receptor.

FIG. 8: Displays the ability of CGRP to inhibit ¹²⁵I-CGRP binding in NIH/3T3 cells stably expressing the Skb-CGRP receptor.

FIG. 9: Displays the ability of CGRP to inhibit ¹²⁵I-CGRP binding in NIH/3T3 untransfected control cells.

FIG. 10: Molecular structure of the JPr-CGRP receptor.

FIG. 11: Alignment of Amino Acid Sequences for the JPr-CGRP-R, rat Adrenomedullin, and hCGRP-R.

FIG. 12: Alignment of Amino Acid Sequences for the JPr-CGRP (top), dog RDC-1 (dog CGRP-R), and human CGRP-R (bottom).

FIG. 13: Displays in situ hybridization of JPr-CGRP receptors in rat brain cortex with the JPr-CGRP-R cRNA probe.

FIG. 14: Displays in situ hybridization of JPr-CGRP receptors in rat brain hippocampus with the cRNA probe.

FIG. 15: Displays in situ hybridization of JPr-CGRP receptors in rat Purkinje fibers with the cRNA probe.

FIG. 16: Displays in situ hybridization of JPr-CGRP receptors in rat spinal cord with the cRNA probe.

FIG. 17: Displays an agarose electrophoresis gel testing for presence of RAMP in cell preps used in CGRP receptor testing herein.

### DETAILED DESCRIPTION

Introduction

CGRP and CGRP receptor proteins are involved in a number of vitally important physiological processes (e.g., ranging from involvement in postmenopausal bone loss, to vasodilation, migraines, chronic pain, diabetes, inflammation, cancer, obesity, Paget's disease, vomiting, benign prostatic hypertrophy, depression, psychosis, allergies, asthma, ulcers, angina pectoris, acute heart failure, hypotension, urinary retention, myocardial infarction, etc.). These numerous involvements emphasize the importance of CGRP and its receptor (or receptors) in further development of therapeutic and/or prophylactic treatments.

The present invention includes sequences (both nucleic acid and amino acid), methods, and compositions comprising such CGRP receptor molecules. The present invention provides isolated or recombinant nucleic acids (including, e.g., those listed as SEQ ID NO:1-2, sequences which encode, e.g., the polypeptides of SEQ ID NO:3-4, nucleic acid sequences which hybridize under highly stringent conditions to the above, and fragments of any of these sequences which encode a calcitonin gene related peptide (CGRP) receptor, optionally from a rat or a human, and/or which bind CGRP or CGRP-like molecules. The present invention also provides methods for generating, methods for identifying, methods of use (e.g., in therapeutic/prophylactic treatment of conditions), and compositions comprising the above sequences.

Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an” and “the” include plural referents unless the content clearly dictates otherwise. Thus, for example, reference to “a molecule” optionally includes a combination of two or more such molecules, and the like.

Unless defined otherwise, all scientific and technical terms are understood to have the same meaning as commonly used in the art to which they pertain. For the purpose of the present invention, the following terms are defined below.

As used herein, proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 91%, 92%, 93%, 94%, 95%, 96%, 97%, 98%, 99% or more can also be used to establish homology. Methods for determining sequence similarity percentages (e.g., BLASTP and BLASTN using default parameters) are described herein and are generally publicly available. In some embodiments a homologous protein can comprise a reporter moiety attached to the homologous sequence. For example, any naturally occurring nucleic acid can be modified by any available mutagenesis method to include one or more additional, alternative, or altered sequence (e.g., a sequence encoding a selective marker, etc.). When expressed, this mutagenized nucleic acid encodes a polypeptide comprising such selective marker. A mutation process can, of course, additionally alter one or more nucleic acid sequence, thereby changing one or more amino acid in the resulting mutant protein as well.

The term “derived from” refers to a component that is isolated from, or isolated and modified, or generated, e.g., chemically synthesized, using information of the component.

The term “nucleic acid” as used herein is generally used in its typical art-recognized meaning to refer to a ribose nucleic acid (RNA) or a deoxyribose nucleic acid (DNA) polymer or analog thereof, e.g., a nucleotide polymer comprising modifications of the nucleotides, a peptide nucleic acid (PNA), or the like. In certain applications, the nucleic acid can be a polymer including both RNA and DNA subunits. A nucleic acid can be, e.g., a chromosome or chromosomal segment, a vector (e.g., an expression vector), a naked DNA or RNA polymer, the product of a polymerase chain reaction (PCR), an oligonucleotide, a probe, etc.

The term “polynucleotide sequence” refers to a contiguous sequence of nucleotides in a single nucleic acid or to a representation, e.g., a character string, thereof, depending on context.

The term “amino acid sequence” refers to a polymer of amino acids (e.g., a protein, polypeptide, etc.) or to a character string representing an amino acid polymer, depending on context.

“Substantially the entire length” of a polynucleotide or amino acid sequence typically refers to at least about 80%, at least about 90%, at least about 91%, at least about 92%, at least about 93%, at least about 94%, at least about 95%, at least about 96%, at least about 97%, at least about 98%, at least about 99% or more of a length of an amino acid or nucleic acid sequence.

A nucleic acid, protein, peptide, polypeptide, or other similar component is “isolated” when it is partially or completely separated from components with which it is normally associated such as other peptides, polypeptides, proteins (including complexes, e.g., polymerases and ribosomes which may accompany a native sequence), nucleic acids, cells, synthetic reagents, cellular contaminants, or cellular components, etc. For example, a component is isolated when it is separated from other components with which it is normally associated in a cell from which it was originally derived. A nucleic acid, polypeptide, or other component is substantially pure when it is partially or completely recovered or separated from other components of its natural environment (e.g., as in a cell, etc.) such that it is the predominant species present in a composition, mixture, or collection of components (e.g., the component is more abundant than any other individual species in a composition on a molar basis). In some embodiments, such a preparation consists of more than 70%, typically more than 80%, or preferably more than 90%, 91%, 92%, 93%, 94%, 95%, 96%, 97%, 98%, or 99% or more of the isolated species.

In some aspects, a “substantially pure” or “isolated” nucleic acid (e.g., RNA or DNA), polypeptide, protein, or composition also means wherein the object species (e.g., nucleic acid or polypeptide, etc.) comprises at least about 50%, 60%, 70%, 80%, 90%, 95%, or 99% or more by weight (or on a molar basis) of all macromolecular species present. A substantially pure or isolated composition can also comprise at least about 80%, 90%, 95%, 96%, 97%, 98%, or 99% or more by weight of all macromolecular species present in the composition. An isolated object species can also be purified to essential homogeneity (e.g., contaminant species cannot be detected in the composition by conventional detection methods) wherein the composition consists essentially of derivatives of a single macromolecular species.

The term “isolated nucleic acid” can also refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector (e.g., an expression vector); integrated into the genome of the same or of a different species than the organism, including, e.g., an organism, from which it was originally derived; linked to an additional coding sequence to form a hybrid gene encoding a chimeric polypeptide; or independent of any other DNA sequences. Such DNA may be double-stranded or single-stranded, sense or antisense.

A nucleic acid or polypeptide is “recombinant” when it is artificial or engineered, or derived from an artificial or engineered protein or nucleic acid. The term “recombinant” when used with reference e.g., to a cell, nucleotide, vector, or polypeptide typically indicates that the cell, nucleotide, or vector has been modified by the introduction of a heterologous (or foreign) nucleic acid or the alteration of a native nucleic acid, or that the polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g., genes) that would be abnormally expressed, under-expressed, or not expressed at all.

The term “recombinant nucleic acid” (e.g., DNA or RNA) indicates, for example, that a nucleotide sequence is not naturally occurring or is made by the combination (for example, artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. The term “recombinantly produced” refers to an artificial combination usually accomplished by either chemical synthesis means, recursive sequence recombination of nucleic acid segments or other diversity generation methods of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated. A “recombinant polypeptide” or “recombinant protein” usually refers to polypeptide or protein, respectively, that results from a cloned or recombinant gene or nucleic acid.

A “vector,” as used herein, optionally comprises, e.g., plasmids, cosmids, viruses, fragments of viruses, YACs, etc. An “expression vector” is a vector, e.g., a plasmid, capable of producing transcripts and, potentially, polypeptides encoded by a polynucleotide sequence. Typically, an expression vector is capable of producing transcripts in an exogenous cell, e.g., a bacterial cell, a mammalian cultured cell, or a mammalian cell, etc. Expression of a product can be either constitutive or inducible depending, e.g., on the promoter selected. In the context of an expression vector a promoter is “operably linked” to a polynucleotide sequence if it is capable of regulating expression of the associated polynucleotide sequence. The term also applies to alternative exogenous gene constructs, such as expressed or integrated transgenes. Similarly, the term operably linked applies equally to alternative or additional transcriptional regulatory sequences such as enhancers, associated with a polynucleotide sequence.

The term “subject” as used herein includes, but is not limited to, a mammal, including, e.g., a human, non-human primate (e.g., a monkey), mouse, pig, cow, goat, rabbit, rat, guinea pig, hamster, horse, monkey, sheep, or other non-human mammal, a non-mammal, including, e.g., a non-mammalian vertebrate, such as a bird (e.g., a chicken or duck) or a fish; an a non-mammalian invertebrate. In some embodiments, the methods and compositions, etc., of the invention are used to treat (both prophylactically and/or therapeutically) human or non-human animals.

The term “pharmaceutical composition” herein means a composition suitable for pharmaceutical use in or administration to a subject, including an animal or human. A pharmaceutical composition generally comprises an effective amount of an active agent (e.g., the CGRP receptor, or a portion thereof, of the invention) and a pharmaceutically acceptable carrier (e.g., a buffer, adjuvant, or the like).

The term “effective amount” means a dosage or amount sufficient to produce a desired result. The desired result may comprise an objective or subjective improvement in the recipient of the dosage or amount (e.g., long-term survival, alteration in gastrointestinal activity, effective prevention of a disease state, etc.).

The term “ligand” as used herein, refers to a molecule which is capable of binding to a receptor (or other similar molecule), thus optionally forming a ligand-receptor complex. For example, a CGRP molecule acts as a ligand to a CGRP receptor. Ligands are optionally “agonists,” meaning that the molecule binds to a receptor, thus forming a complex, and elicits or causes a response specific of the nature of the receptor involved. For example, a [Cys(ACM)2,7]hCGRP molecule can act as an agonist to a CGRP receptor eliciting specific responses from the receptor. An “antagonist” refers to a compound that binds to a receptor, thus forming a complex, but which does not elicit a response from the receptor or which elicits a different response (e.g., in type or level—typically a lower level of response) than the response typically elicited by binding of an agonist of the receptor. For example, CGRP (8-37) can act as an antagonist to CGRP receptors and, even though it binds to the receptor, does not elicit a response from the receptor. For example, the agonist/antagonist (i.e., modulatory) effect of a molecule binding to a CGRP-R is optionally determined through measurement of CAMP. See, Example 1 below. Also as used herein, “modulating the activity” of a receptor (e.g., a CGRP receptor) refers in general to agonist or antagonist action on, or through, a receptor. Accordingly, a “modulator” or the like herein acts in general as an agonist or antagonist on, or through, a receptor.

A “CGRP molecule” comprises a calcitonin gene related peptide. As defined above, CGRP comprises a 37-amino acid neuropeptide encoded as a splice variant by the calcitonin/CGRP gene. See, e.g., S. G. Amara, et al. (1982) Nature 298:240-244 and M. G. Rosenfeld, et al. (1983) Nature 204:129-135. CGRP peptides are structurally similar to members of the Adrenomedullin and Amylin families (e.g., including similar N-terminal disulfide bonds and C-terminal amide). Two biologically active forms of CGRP (α-CGRP and β-CGRP) exist in both rats and humans and induce similar biological activities. CGRP receptors (e.g., receptors that bind CGRP and/or CGRP derivatives or CGRP-like molecules) are highly expressed throughout the brain and gastrointestinal tract where they are involved in, e.g., regulation of pain responses and gut motility. A CGRP receptor herein typically refers to an isolated (or cell associated) receptor which displays CGRP binding activity. Also, depending upon context herein, CGRP receptor can also include fragments or portions of a full length (or full sized, complete, etc.) CGRP receptor. CGRP has also been found to be involved in numerous important physiological activities, such as: vasodilatation, cardiac acceleration, inhibition of gastric acid secretion, reduction of intestinal motility, regulation of glucose metabolism, diminution of appetite, and reduction of growth hormone release, etc. See, e.g., S. J. Wimalawansa, (1997) Crit Rev in Neurobiol 11, 167-239. A “CGRP-like molecule” optionally comprises an antagonist or an agonist (see, above) to the CGRP receptor. For example, two non-limiting examples include CGRP(8-37) (which optionally acts as an antagonist to the CGRP-receptor) and [Cys(ACM)2,7]hCGRP (which optionally acts as an agonist to the CGRP receptor). See, e.g., U.S. Pat. No. 6,268, 474 for descriptions of additional CGRP-receptor agonists/antagonists. Other CGRP-like molecules optionally comprise CGRP molecules from different species (e.g., from species other than the one whose CGRP-receptor is used to screen/bind/etc. the CGRP-like molecule). Additionally, other CGRP-like molecules need not be polypeptides. In other words, such non-peptide CGRP-like molecules will still bind to the CGRP-receptor (e.g., optionally as an agonist or an antagonist). Other CGRP-like molecules are optionally screened for by the methods and compositions, etc. of the invention, see, below.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk or likelihood or probability of developing the disease, pathology, or medical disorder. A prophylactic treatment functions as a preventative treatment against a disease or disorder. A “prophylactic activity” is an activity of an agent, such a CGRP receptor protein (or portion thereof), or composition thereof, that, when administered to a subject who does not display signs or symptoms of a pathology, disease or disorder (or who displays only early signs or symptoms of a pathology, disease, or disorder) diminishes, prevents, or decreases the risk/likelihood/probability of the subject developing the pathology, disease, or disorder. A “prophylactically useful” agent or compound (e.g., a CGRP receptor protein of the invention or a portion thereof) refers to an agent or compound that is useful in diminishing, preventing, treating, or decreasing development of a pathology, disease or disorder.

A “therapeutic treatment” is a treatment administered to a subject who displays symptoms or signs of a pathology, disease, or disorder, in which treatment is administered to the subject for the purpose of diminishing or eliminating those signs or symptoms of pathology, disease, or disorder (e.g., and, thus, optionally diminishes or eliminates the underlying cause(s) of the pathology, disease, or disorder). A “therapeutic activity” is an activity of an agent, such a CGRP receptor protein of the invention or a portion thereof, or composition thereof, that eliminates or diminishes signs or symptoms of a pathology, disease or disorder, when administered to a subject suffering from such signs or symptoms. A “therapeutically useful” agent or compound (e.g., a CGRP receptor protein of the invention or a portion thereof, or a ligand bound to the CGRP receptor (e.g., a ligand/receptor complex) that exhibits pharmacological activity) indicates that an agent or compound is useful in diminishing, treating, or eliminating such signs or symptoms of the pathology, disease or disorder.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (V_{H}) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab′)₂, a dimer of Fab which itself is a light chain joined to V_{H}-C_{H}1 by a disulfide bond. The F(ab′)₂ may be reduced under mild conditions to break the disulfide linkage in the hinge region thereby converting the F(ab′)₂dimer into an Fab′ monomer. The Fab′ monomer is essentially an Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments, etc. may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv or scFv) antibodies in which a variable heavy and a variable light chain are joined together (directly or through a peptide linker) to form a continuous polypeptide.

Discussion

The present invention relates to sequences for CGRP receptors, as well as methods and compositions for such. In some embodiments, the current invention comprises an isolated or recombinant nucleic acid encoding a CGRP receptor (e.g., in an expression vector), that comprises a sequence selected from: the sequence of SEQ ID NO:1 or 2 (or a complementary polynucleotide sequence thereof), a polynucleotide sequence (or a complementary polynucleotide sequence thereof) encoding the polypeptide sequence of SEQ ID NO:3-4, a polynucleotide sequence that hybridizes under highly stringent conditions over substantially the entire length of the above sequences, and a fragment of the above sequences that encodes a polypeptide that binds a CGRP molecule or a CGRP-like molecule.

Polynucleotide sequences of the invention include, e.g., the polynucleotide sequences represented by SEQ ID NO:1 and SEQ ID NO: 2. In addition to the sequences expressly provided in the accompanying sequence listing, polynucleotide sequences that are highly related both structurally and functionally are polynucleotides of the invention are included (e.g., polynucleotides encoding CGRP-receptor proteins), which proteins have greater than 91%, etc. sequence identity to SEQ ID NO:3 or to SEQ ID NO:4. Polynucleotides encoding a polypeptide and having a sequence or subsequence encoded by SEQ ID NO:1 or SEQ ID NO: 2, or subsequences thereof are one embodiment of the invention. In addition, polynucleotide sequences of the invention include polynucleotide sequences that hybridize under stringent conditions to a polynucleotide sequence comprising either of SEQ ID NO: 1 or SEQ ID NO: 2.

In addition to the polynucleotide sequences of the invention, e.g., enumerated in SEQ ID NO: 1 to SEQ ID NO: 2, polynucleotide sequences that are substantially identical to a polynucleotide of the invention are features of the invention and can be used in the compositions and methods of the invention. Substantially identical, or substantially similar polynucleotide (or polypeptide) sequences are defined as polynucleotide (or polypeptide) sequences that are identical, on a nucleotide by nucleotide basis (and/or on an amino acid by amino acid basis), with at least a subsequence of a reference polynucleotide (or polypeptide), e.g., selected from SEQ ID NO: 1-2 (or 3-4 in the case of amino acids). Such polynucleotides can include, e.g., insertions, deletions, and substitutions relative to any of SEQ ID NO: 1-2 for polynucleotides and SEQ ID NO: 3-4 for amino acids. For example, such polypeptides are typically at least about 91% identical to a reference polypeptide selected from among SEQ ID NO: 3 and SEQ ID NO: 4 which is isolated or recombinant and optionally binds CGRP and/or a CGRP-like molecule. That is, at least greater than 9 out of 10 amino acids within a window of comparison are identical to the reference sequence selected SEQ ID NO: 3-4 (or 1-2 for a comparable sequence identity comparison done for polynucleotides). Frequently, such sequences are at least about 91%, usually at least about 92%, and often at least about 93%, or even at least about 94%, or about 95%, 96%, 97%, 98%, or 99%, or more identical to the reference sequence, e.g., at least one of SEQ ID NO: 3 or SEQ ID NO: 4.

Subsequences of the polynucleotides of the invention described above, e.g., SEQ ID NO: 1-2, including at least 650 contiguous nucleotides or complementary subsequences thereof are also a feature of the invention. More commonly a subsequence includes at least 700, e.g., of one or more of SEQ ID NO: 1 through SEQ ID NO: 2. Typically, the subsequence includes at least 750, frequently at least 800, at least 850, at least 900, at least 950, at least 1000, and usually at least 1080, 1085, 1086, 1087, 1088, or 1089 or more contiguous nucleotides of one of the specified polynucleotide sequences. Such subsequences can be, e.g., oligonucleotides, such as synthetic oligonucleotides, or full-length genes or cDNAs.

Characterization of CGRP receptors of the invention

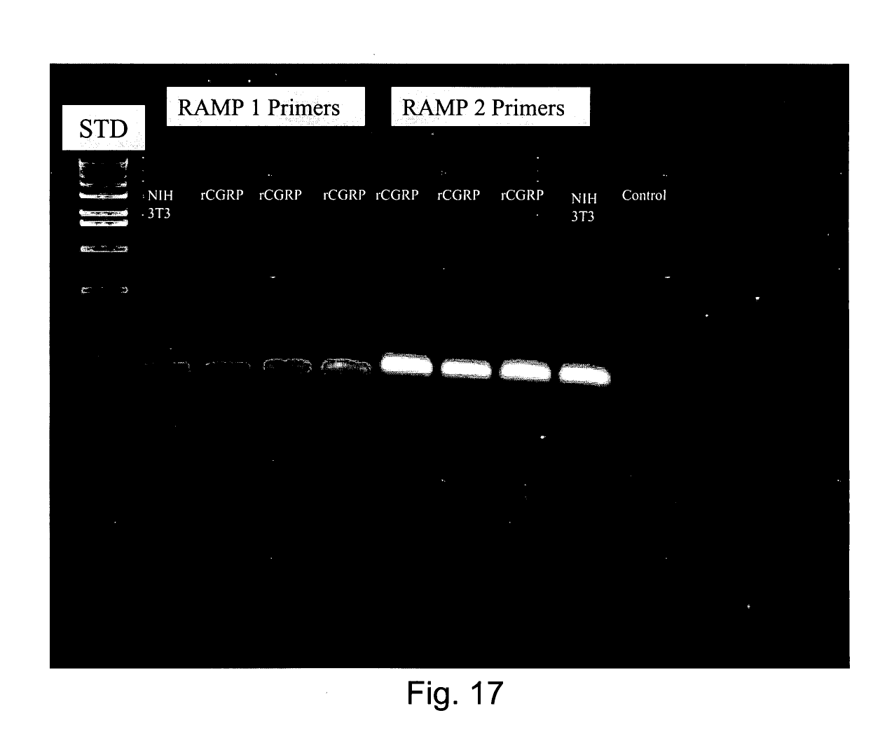

The current invention comprises, in some aspects, a rat CGRP receptor (JPr-CGRP-R, also described herein as rat CGRP-R, rCGRP-R, or CGRP receptor) that exhibits high homology with the dog RDC-1 receptor and low similarity with the previously reported human CGRP receptor, i.e., Skb-CGRP-R. In other aspects, the current invention comprises a human homologue of the JPr-CGRP-R, namely JPh-CGRP-R (as opposed to previous human CGRP receptors such as, e.g., Skb-CGRP-R). The human homologue JPh-CGRP-R herein is, thus, thought to exhibit similar characterization of the rat CGRP receptor homologue (i.e., JPr-CGRP-R). The JPr-CGRP-R protein is a 362 amino acid protein and has an 85% homology to the rat Adrenomedullin receptor and a calculated molecular mass of 40 KD. In some embodiments, the JPr-CGRP receptor does not require cotransfection with RAMP proteins for full pharmacological expression. FIG. 17 displays that RAMP was actually not present (or optionally not expressed) in cells utilized in the Examples herein with JPr-CGRP-R. Additionally, Radioligand Binding Inhibition assays (see, below) indicate that a high affinity interaction exists between CGRP and the JPr-CGRP receptor of the invention. Adenylate Cyclase Stimulation assays also reveal an intracellular cAMP response that is dependent upon the relative concentration of added CGRP.

SEQ ID NO:1 represents the nucleic acid sequence of JPr-CGRP-R, while SEQ ID NO:3 represents the corresponding amino acid sequence of 362 amino acids. It will be appreciated that as listed in the sequence table herein, the nucleic acid sequence of the JPr-CGRP-R of the invention comprises both 5′ and 3′ untranslated sequences around the sequence encoding the 362 amino acid JPr-CGRP-R protein. Thus in SEQ ID NO:1 the first 38 nucleotides and the final 119 nucleotides represent untranslated regions, and the intervening 1089 nucleotides encode the 362 amino acid polypeptide (plus a stop codon). It is to be understood that, depending upon context, as referenced herein SEQ ID NO:1 will include the untranslated regions or will not include the untranslated regions around the 362 amino acid encoding region.

Other CGRP receptors (e.g., those characterized by Nambi Aiyar et al. of SmithKline Beecham Pharmaceuticals, Skb-CGRP-R) comprise a different structure at the cDNA level than the JPr-CGRP-R of the invention and the JPh-CGRP-R of the invention. Cf., Nambi Aiyar et al., “A cDNA Encoding the Calcitonin Gene-related Peptide Type 1 Receptor” J Bio Chem (1996) 19:11325-11329. As shown herein, the expression pattern, pharmacology, and signal transduction properties of these two receptors types (i.e., those represented by Skb-CGRP and those represented by JPr-CGRP-R and its human homologue, sometimes referred to herein as JPh-CGRP-R), and their relationship to each other and CGRP, are quite distinct. As described in more detail below, the JPr-CGRP-R cDNA and Skb-CGRP-R cDNA were both transfected into NIH/3T3 cells. In order to reveal the gene expression pattern of the JPr-CGRP receptor, Reverse Transcriptase-Polymerase Chain Reaction (RT-PCR) studies were performed on such cells, as well as on human SK-N-MC neuroblastoma cells believed to contain endogenous CGRP receptors. To elucidate the pharmacology and signal transduction properties of the two receptor types, parallel Binding Inhibition and Adenylate Cyclase assays were performed on each cell line. Again, as seen in more detail below, JPr-CGRP-R mRNA expression is limited only to cells transfected with such gene. Furthermore, while both receptors interact with the CGRP peptide, the JPr-CGRP receptor of the invention (i.e., the receptor corresponding to the type comprising JPr-CGRP-R and JPh-CGRP-R as opposed to Skb-CGRP-R), does so with higher specificity and affinity.

Prior to the cloning of the JPr-CGRP-R gene of the invention, at least two CGRP receptor subtypes had been described by pharmacological studies on brain membrane preparations and in tumor cell lines expressing CGRP receptors. The two subtypes differed in their affinities for the ligands CGRP, CGRP(8-37), and [Cys(ACM)2,7]hCGRP. CGRP(8-37), for example, lacks 7 N-terminal amino acid residues and is a partial antagonist of CGRP receptors. Pharmacological studies have shown CGRP(8-37) to be a selective antagonist of CGRP type 1 receptors. On the other hand, CGRP(8-37) has a very low affinity for CGRP type 2 receptors. Such differing affinity indicates the existence of a least two unique CGRP-R subtypes. Previously, one type of CGRP receptor has been investigated as an orphan calcitonin- like receptor in rats and humans. An orphan CGRP receptor had also been investigated in dogs (RDC-1). However, an interaction between these receptors and CGRP or any other ligand was not clearly demonstrated. See, also, Heesen, et al., “Cloning and chromosomal mapping of an orphan chemokine receptor: mouse RDC1,”Immunogenetics (1998) 47:364-370.

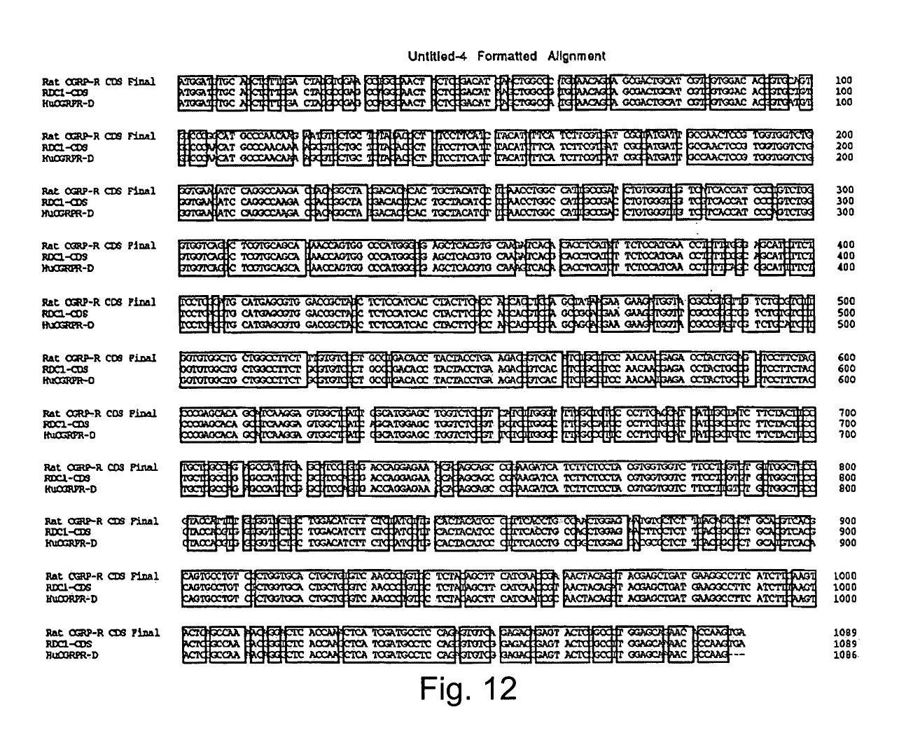

However, following the above characterization attempts, the expression of CGRP receptors by molecular means was demonstrated. Nambi Aiyar et al. of SmithKline Beecham Pharmaceuticals reported cloning of human and porcine CGRP type 1 receptors (Skb-CGRP-R), and functional expression of such in HEK 293 cells. It was later shown by Foord et al., that RAMP cotransfection was required for full expression of the Skb-CGRP-R vector. See, “RAMPs regulate the transport and ligand specificity of the Calcitonin-receptor-like receptor” (1998) Nature 393:333-339. The inventors have cloned a CGRP receptor encoding gene (JPr-CGRP-R) and functionally expressed it in NIH/3T3 cells (see, below and elsewhere herein). A comparison of the cDNA sequence of both the genes of the current invention (i.e., JPr-CGRP-R and JPh-CGRP-R) against the work of Nambi Aiyar et al. reveal dramatic differences. For example, the JPr-CGRP-R gene of the invention encodes a 362 amino acid protein with a strong sequence homology to the rat Adrenomedullin receptor (85%), as well as to the dog RDC-1 receptor. See, e.g., FIG. 12. In contrast, the Skb-CGRP-R gene encodes a 461 amino acid protein sharing its strongest sequence homology with the human Calcitonin receptor (55.5%). Both the CGRPs of the invention and the receptor characterized by Nambi Aiyar et al. share characteristics consistent with other G-protein coupled receptors of the same protein family (e.g., both contain seven hydrophobic regions of approximately 16 to 28 amino acids that likely represent the seven-transmembrane motif found among G-protein coupled receptors, both types encode receptors that interact with the CGRP protein, Adenylate Cyclase Stimulation and Binding Inhibition assays for both receptor types reveal responses dependent on the concentration of CGRP, etc.).

Some studies have proposed the existence of receptor associated modifying proteins (RAMP) which interact with CGRP receptor-like receptors to confer unique pharmacological properties. See, e.g., Drake, W. M. et al., “Desensitization of CGRP and Adrenomedullin Receptors in KS-N-MC Cells: Implications for the RAMP Hypothesis” Endocrinology (1999) 140:533-536. These single transmembrane domain proteins appear to be required for glycosylation and transport of the CGRP receptors to the plasma membrane. They are also involved in ligand binding and specificity. When the protein RAMP₁ interacts with a Calcitonin receptor-like receptor, it confers CGRP receptor pharmacology. When RAMP₂ is present, it confers Adrenomedullin pharmacology. The JPr-CGRP/JPh-CGRP and Skb-CGRP receptors' unique pharmacology thus is optionally influenced by interactions with RAMP proteins. These proteins might be co-expressed within a cell's plasma membrane, influencing the receptors' tertiary structure and sensitivity to CGRP, thus, optionally providing cells with greater flexibility in manipulating the responses of their receptors.

RAMP proteins may play a role in establishing the identity and pharmacological characteristics of Calcitonin receptor-like receptors (CRLRs), therefore it is therefore a consideration whether particular cells naturally express RAMP proteins capable of interacting with the JPr-CGRP-R/JPh-CGRP-R or the Skb-CGRP-R. If RAMP proteins are expressed and affecting receptor function, it is possible that these receptors might behave differently in various host cells containing different sets of endogenous RAMP proteins. In other words, the differing cell lines used (e.g., NIH/3T3, HEK-293, etc.) could optionally influence the pharmacological activity (e.g., binding differences between different CGRP receptors and different agonists/antagonists).

RT-PCR studies indicate the JPr-CGRP receptor of the invention is not expressed natively within cells transfected with the Skb-CGRP receptor. Only cells specifically transfected with the JPr-CGRP-R cDNA responded positively. Thus, responses elicited by the addition of CGRP to Skb-CGRP-R transfected cells are independent of the JPr-CGRP receptor. The receptors themselves must account for the observed pharmacological differences since both genes were transfected into the same strain of cells (NIH/3T3) (see, below). Therefore, the Skb-CGRP-R gene represents a CGRP receptor variant different from the JPr-CGRP-R/JPh-CGRP-R genes of the invention. This is not uncommon since it is quite possible for multiple receptor subtypes specific for a single protein to exist.

SK-N-MC and HCT8 are non-transgenic cell lines used to probe for in vivo expression of the JPr-CGRP receptor (see, below). For both cells, RT-PCR indicated no native expression of this receptor. Thus, while it is possible that the JPr-CGRP receptor variant is not expressed in either cell, additional complexities exist which suggest alternative explanations for the negative responses. For example, it is possible that a gene similar to the JPr-CGRP receptor is expressed in these cells, but that the sequence of such similar gene (in the regions recognized by the RT-PCR primers) is different enough to inhibit primer binding. Although their cDNA is present, these genes would not be amplified by PCR. Such scenario is possible because the receptors exist in different species. The SK-N-MC and HCT8 are human cells, while the JPr-CGRP receptor originated from rat cells. It is possible that the evolutionary importance of the JPr-CGRP receptor might conserve its endogenous expression across species. Additionally, since both SK-N-MC and HCT8 are cancer cells, it is reasonable to suspect possible defects in gene expression. If the JPr-CGRP receptor's normal expression pattern is corrupted due to mutation, its MRNA will not be present for reverse transcription during RT-PCR.

Radioligand Binding Inhibition assays have further distinguished the properties of the receptor type of the invention and of the Skb-CGRP receptor. Multiple experiments have placed the IC₅₀ value for the JPr-CGRP and Skb-CGRP receptors at 3.98×10⁻⁸ mM and 5.01×10⁻⁷ mM respectively. A lower IC₅₀ value indicates more potent CGRP binding for the JPr-CGRP receptor. This result indicates that the JPr-CGRP receptor of the invention (and optionally the JPh-CGRP-R of the invention) has a greater affinity for CGRP than the Skb-CGRP receptor does. The amino acid sequence and tertiary structure of both CGRP and the JPr-CGRP receptor contribute to this strong interaction.

Adenylate Cyclase Stimulation assays (see, below) indicate two generalizations regarding the two receptors (i.e., the JPr-CGRP/JPh-CGRP receptors of the invention and the Skb-CGRP receptor). First, these receptors participate in signal transduction cascades leading to cAMP production. The most common route is through an intracellular G-protein and the enzyme Adenylate Cyclase. Second, the assays confirm an interaction between the receptors (again, the JPr-CGRP receptor type of the invention and the Skb-CGRP receptor) and CGRP. Multiple experiments place the EC₅₀ value for the JPr-CGRP and Skb-CGRP receptors at 6.46×10⁻⁸ mM and 8.32×10⁻⁸ mM respectively. Thus, a 29% larger CGRP concentration is required for the Skb-CGRP receptor to stimulate cAMP production to 50% of its maximum value. Therefore, the JPr-CGRP receptor produces the more efficacious cAMP stimulation in response to CGRP. Such difference is optionally due to a higher degree of interaction and complementation between the JPr-CGRP-R and CGRP protein amino acid sequences. Additionally, the JPr-CGRP receptor consistently displays a larger cAMP response than the Skb-CGRP receptor at all CGRP concentrations, see, below. Thus, the JPr-CGRP receptor more potently stimulates cAMP in response to CGRP protein. Collectively, these results indicate the JPr-CGRP receptor to be more specific for CGRP. However, both genes may still code for receptors responsive to CGRP stimulation. For example, the two forms could be expressed at different times in vivo. When a sensitive measurement of CGRP concentration is required, the cell might express the JPr-CGRP-R/JPh-CGRP-R variant type. When only a coarse measurement is needed, the Skb-CGRP-R type might be expressed.

The Skb-CGRP receptor transfected into HEK 293 cells receptor showed a 60-fold increase in cAMP production following exposure to maximal concentrations of CGRP. See, Nambi Aiyar et al., above. However, human Calcitonin also stimulated cAMP accumulation in vector control transfected cells to 75% of the maximum amount of cAMP induced by CGRP in the Skb-CGRP-R transfected cells. Since Calcitonin has cross reactivity with the CGRP receptor, it is possible that HEK 293 cells natively express CGRP receptors. This could possibly account for the high levels of observed cAMP accumulation.

As illustrated in the Example below and the discussion above, the JPr-CGRP-R/JPh-CGRP-R and Skb-CGRP-R genes and encoded proteins display low similarity and share different, although related, pharmacological properties. For example, both types interact with the CGRP protein, and both are CGRP receptor types. However, the JPr-CGRP receptor's more efficacious and potent cAMP stimulation, as well as its higher affinity CGRP binding, demonstrate its greater specificity for CGRP. Also, the JPr-CGRP receptor shows response to a wide variety of related peptides such as Amylin, Adrenomedullin, and Calcitonin, although CGRP elicited the most specific interaction.

Probes

In some embodiments, nucleic acids including one or more polynucleotide sequence of the invention are favorably used as probes for the detection of corresponding or related nucleic acids in a variety of contexts, such as in nucleic hybridization experiments, e.g., to find and/or characterize homologous CGRP receptors (e.g., homologues to SEQ ID NO:1) in other species. The probes can be either DNA or RNA molecules, such as restriction fragments of genomic or cloned DNA, cDNAs, PCR amplification products, transcripts, and oligonucleotides, and can vary in length from oligonucleotides as short as about 10 nucleotides in length to chromosomal fragments or cDNAs in excess of 1 kb or more. For example, in some embodiments, a probe of the invention includes a polynucleotide sequence or subsequence selected, e.g., from among SEQ ID NO: 1 or SEQ ID NO: 2, or sequences complementary thereto. Alternatively, polynucleotide sequences that are variants of one of the above-designated sequences are used as probes. Most typically, such variants include one or a few conservative nucleotide variations. For example, pairs (or sets) of oligonucleotides can be selected, in which the two (or more) polynucleotide sequences are conservative variations of each other, wherein one polynucleotide sequence corresponds identically to a first allele or allelic variant and the other(s) corresponds identically to additional alleles or allelic variants. Such pairs of oligonucleotide probes are particularly useful, e.g., for allele specific hybridization experiments to detect polymorphic nucleotides or to, e.g., detect homologous CGRP receptors, e.g., homologous to JPr-CGRP-R, in other species. In other applications, probes are selected that are more divergent, that is probes that are at least about 91% (or about 92%, about 93%, about 94%, about 95%, about 96%, about 97%, about 98%, or about 99% or more ) identical are selected.

The probes of the invention, e.g., as exemplified by sequences derived from SEQ ID NO: 1, can also be used to identify additional useful polynucleotide sequences according to procedures routine in the art. In one set of embodiments, one or more probes, as described above, are utilized to screen libraries of expression products or chromosomal segments (e.g., expression libraries or genomic libraries) to identify clones that include sequences identical to, or with significant sequence similarity to, e.g., one or more probe of SEQ ID NO: 1, i.e., allelic variants, homologues, etc. It will be understood that in addition to such physical methods as library screening, computer assisted bioinformatic approaches (optionally through use of the Internet), e.g., BLAST and other sequence homology search algorithms, and the like, can also be used for identifying related polynucleotide sequences. Polynucleotide sequences identified in this manner are also a feature of the invention.

Oligonucleotide probes are optionally produced via a variety of methods well known to those skilled in the art. Most typically, they are produced by well known synthetic methods, such as the solid phase phosphoramidite triester method described by Beaucage and Caruthers (1981) Tetrahedron Letts 22(20):1859-1862, e.g., using an automated synthesizer, or as described in Needham-VanDevanter et al. (1984) Nucl Acids Res, 12:6159-6168. Oligonucleotides can also be custom made and ordered from a variety of commercial sources known to persons of skill. Purification of oligonucleotides, where necessary, is typically performed by either native acrylamide gel electrophoresis or by anion-exchange HPLC as described in Pearson and Regnier (1983) J Chrom 255:137-149. The sequence of the synthetic oligonucleotides can be verified using the chemical degradation method of Maxam and Gilbert (1980) in Grossman and Moldave (eds.) Academic Press, New York, Methods in Enzymology 65:499-560. Custom oligos can also easily be ordered from a variety of commercial sources known to persons of skill.

In other circumstances, e.g., relating to functional attributes of cells or organisms expressing the polynucleotides and polypeptides of the invention (e.g., the JPr-CGRP receptors of the invention), probes that are polypeptides, peptides or antibodies are favorably utilized. For example, isolated or recombinant polypeptides, polypeptide fragments and peptides derived from any of the amino acid sequences of the invention and/or encoded by polynucleotide sequences of the invention, e.g., selected from SEQ ID NO: 1 or SEQ ID NO: 3, are favorably used to identify and isolate antibodies or other binding proteins, e.g., from phage display libraries, combinatorial libraries, polyclonal sera, and the like.

Antibodies specific for any a polypeptide sequence or subsequence, e.g., of SEQ ID NO: 3, and/or encoded by polynucleotide sequences of the invention, e.g., selected from SEQ ID NO: 1, are likewise valuable as probes for evaluating expression products, e.g., from cells or tissues. In addition, antibodies are particularly suitable for evaluating expression of proteins comprising amino acid subsequences, e.g., of SEQ ID NO: 3, or encoded by polynucleotides sequences of the invention, e.g., selected from SEQ ID NO:1, in situ, in a tissue array, in a cell, tissue or organism, e.g., an organism providing an experimental model CGRP binding to CGRP receptors. Antibodies can be directly labeled with a detectable reagent, or detected indirectly by labeling of a secondary antibody specific for the heavy chain constant region (i.e., isotype) of the specific antibody. Additional details regarding production of specific antibodies are provided below.

Vectors, Promoters and Expression Systems

The present invention includes recombinant constructs incorporating one or more of the nucleic acid sequences described above, e.g., SEQ ID NO:1 or SEQ ID NO:2, etc. Such constructs optionally include a vector, for example, a plasmid, a cosmid, a phage, a virus, a bacterial artificial chromosome (BAC), a yeast artificial chromosome (YAC), etc., into which one or more of the polynucleotide sequences of the invention, e.g., comprising any of SEQ ID NO: 1 or SEQ ID NO:2, or a subsequence thereof etc., has been inserted, in a forward or reverse orientation. For example, the inserted nucleic acid can include a chromosomal sequence or cDNA including all or part of at least one of the polynucleotide sequences of the invention, e.g., selected from SEQ ID NO: 1 or SEQ ID NO:2, etc. In one embodiment, the construct further comprises regulatory sequences, including, for example, a promoter, operably linked to the sequence. Large numbers of suitable vectors and promoters are known to those of skill in the art, and are commercially available.

The polynucleotides of the present invention can be included in any one of a variety of vectors suitable for generating sense or antisense RNA, and optionally, polypeptide (or peptide) expression products (e.g., a CGRP receptor). Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others (e.g., pCDL). Any vector that is capable of introducing genetic material into a cell, and, if replication is desired, which is replicable in the relevant host can be used.

In an expression vector, the polynucleotide sequence of interest is physically arranged in proximity and orientation to an appropriate transcription control sequence (e.g., promoter, and optionally, one or more enhancers) to direct mRNA synthesis. That is, the polynucleotide sequence of interest is operably linked to an appropriate transcription control sequence. Examples of such promoters include: LTR or SV40 promoter, E. coli lac or trp promoter, phage lambda P_{L} promoter, and other promoters known to control expression of genes in prokaryotic or eukaryotic cells or their viruses. The expression vector also contains, e.g., a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

Additional Expression Elements

Where translation of polypeptide encoded by a nucleic acid comprising a polynucleotide sequence of the invention is desired, additional translation specific initiation signals can improve the efficiency of translation. These signals can include, e.g., an ATG initiation codon and adjacent sequences. In some cases, for example, full-length cDNA molecules or chromosomal segments including a coding sequence incorporating, e.g., a polynucleotide sequence of the invention (e.g., as in SEQ ID NO:1 or SEQ ID NO:2), a translation initiation codon and associated sequence elements are inserted into the appropriate expression vector simultaneously with the polynucleotide sequence of interest. In such cases, additional translational control signals frequently are not required. However, in cases where only a polypeptide coding sequence, or a portion thereof, is inserted, exogenous translational control signals, including an ATG initiation codon is often provided for expression of the relevant sequence. The initiation codon is put in the correct reading frame to ensure transcription of the polynucleotide sequence of interest. Exogenous transcriptional elements and initiation codons can be of various origins, both natural and synthetic. The efficiency of expression can be enhanced by the inclusion of enhancers appropriate to the cell system in use (see, e.g., Scharf D. et al. (1994) Results Probl Cell Differ 20:125-62; Bittner et al. (1987) Methods in Enzymol 153:516-544).

Expression Hosts

The present invention also relates to host cells which are introduced (transduced, transformed or transfected) with vectors of the invention, and the production of polypeptides of the invention by recombinant techniques. Host cells are genetically engineered (i.e., transduced, transformed or transfected) with a vector, such as an expression vector, of this invention. As described above, the vector can be in the form of a plasmid, a viral particle, a phage, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as NIH3T3, Swiss 3T3, COS, CHO, BHK, HEK 293, MCF-7, T-47D, 2329, ZR-75-1, BT-474, SKBR-3, BT-20, MDA-MB-231, CAMA, HCC38, 2336, 2321, 2338, HMEC, MCF-10A, MCF-12A or Bowes melanoma; plant cells, etc.

The engineered host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, 3^{rd} edition, Wiley- Liss, New York and the references cited therein. Expression products corresponding to the nucleic acids of the invention can also be produced in non-animal cells such as plants, yeast, fungi, bacteria and the like. In addition to Sambrook, Berger and Ausubel, all infra, details regarding cell culture can be found in Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla.

In bacterial systems, a number of expression vectors can be selected depending upon the use intended for the expressed product. For example, when large quantities of a polypeptide or fragments thereof are needed for the production of antibodies, vectors which direct high-level expression of fusion proteins that are readily purified are favorably employed. Such vectors include, but are not limited to, multifunctional E. coli cloning and expression vectors such as BLUESCRIPT (Stratagene), in which the coding sequence of interest, e.g., sequences comprising SEQ ID NO:1 or SEQ ID NO: 2, etc., can be ligated into the vector in-frame with sequences for the amino-terminal translation initiating Methionine and the subsequent 7 residues of beta-galactosidase producing a catalytically active beta galactosidase fusion protein; pIN vectors (Van Heeke & Schuster (1989) J Biol Chem 264:5503-5509); pET vectors (Novagen, Madison Wis.); and the like. Similarly, in the yeast Saccharomyces cerevisiae a number of vectors containing constitutive or inducible promoters such as alpha factor, alcohol oxidase and PGH can be used for production of the desired expression products. For reviews, see Ausubel, infra, and Grant et al., (1987); Methods in Enzymology 153:516-544.

In mammalian host cells, a number of expression systems, such as viral-based systems, can be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome will result in a viable virus capable of expressing the polypeptides of interest in infected host cells (Logan and Shenk (1984) Proc Natl Acad Sci 81:3655-3659). In addition, transcription enhancers, such as the rous sarcoma virus (RSV) enhancer, can be used to increase expression in mammalian host cells.

Transformed or transfected host cells containing the expression vectors described above are also a feature of the invention. The host cell can be an eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing, which cleaves a precursor form into a mature form, of the protein is sometimes important for correct insertion, folding and/or function. Additionally proper location within a host cell (e.g., on the cell surface) is also important. Different host cells such as NIH3T3, Swiss 3T3, 3T3, COS, CHO, HeLa, BHK, MDCK, 293, W138, MCF-7, T-47D, 2329, ZR-75-1, BT-474, SKBR-3, BT-20, MDA-MB-231, CAMA, HCC38, 2336, 2321, 2338, HMEC, MCF-10A, MCF-12A, etc. have specific cellular machinery and characteristic mechanisms for such post-translational activities and can be chosen to ensure the correct modification and processing of the current introduced, foreign protein.

For long-term, high-yield production of recombinant proteins encoded by, or having subsequences encoded by, the polynucleotides of the invention, stable expression systems are typically used. For example, cell lines, stably expressing a polypeptide of the invention, are transfected using expression vectors which contain viral origins of replication or endogenous expression elements and a selectable marker gene. For example, following the introduction of the vector, cells are allowed to grow for 1-2 days in an enriched media before they are switched to selective media. The purpose of the selectable marker is to confer resistance to selection, and its presence allows growth and recovery of cells that successfully express the introduced sequences. Thus, resistant clumps of stably transformed cells, e.g., derived from single cell type, can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The cells expressing said protein can be sorted, isolated and/or purified. The protein or fragment thereof produced by a recombinant cell can be secreted, membrane-bound, or retained intracellularly, depending on the sequence and/or the vector used.

In some embodiments of the invention, transgenic animals are optionally produced comprising the CGRP receptors of the invention. Transgenic animals optionally comprise any species, including, but not limited to: non-human primates (e.g., monkeys, chimpanzees, baboons, etc.), cows, mice, rats, rabbits, pigs, goats, sheep, rabbits, etc. Such transgenic organisms produced optionally comprise “over-expression” of one or more CGRP receptor of the invention, e.g., with introduction of several copies of a CGRP receptor gene (or fragments thereof) into the organism with suitable promoters to express the receptor. Optionally, such expression or over-expression is localized to particular cell types and/or tissues within the transgenic organism. In other embodiments, the transgenic organism comprises a “knockout” mutation in the CGRP receptor gene. In other words, a complete or partial suppression of protein expression from endogenous CGRP receptor genes is achieved. For example, in embryonic stem cells, a mutation in the CGRP receptor can lead to inactivation of the CGRP receptor (or alteration in its gene expression). Such transgenic knockout animals are useful to follow the function/activation/etc. of CGRP receptors.

Basically any known technique in the art is optionally used to create transgenic animals comprising the CGRP receptors of the invention. For example, microinjection, embryo electroporation, sperm mediated gene transfer, gene targeting in embryonic stem cells, retrovirus mediated gene transfer into germ lines, etc. are all optional methods of producing transgenic animals comprising the CRGP receptors of the invention. See, e.g., Gordon, Intl Rev Cytol (1989) 115:171-229, Lavitrano, et al. Cell (1989) 57:717-723, Lo, Mol Cell Biol (1983) 3:1803-1814, U.S. Pat. No. 4,873,191, and Van der Putten, et al. Proc Natl Acad Sci, USA (1985) n32:6148-6152, etc.

Polypeptide Production and Recovery

Following transduction of a suitable host cell line or strain and growth of the host cells to an appropriate cell density, the selected promoter is induced by appropriate means (e.g., temperature shift or chemical induction) and cells are cultured for an additional period. In some embodiments, a secreted polypeptide product, e.g., a CGRP receptor as in a secreted fusion protein form, etc., is then recovered from the culture medium. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art. Additionally, cells expressing a polypeptide product of the invention can be utilized without separating the polypeptide from the cell, e.g., as done in screening of CGRP modulatory molecules (see, below). In such situations, the polypeptide of the invention is optionally expressed on the cell surface and is examined thus (e.g., by having CGRP or CGRP-like molecules bind to the polypeptide of the invention on the cell surface). See, Example 1, below. Such cells are also features of the invention.

Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems known to those skilled in the art), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted herein, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

Alternatively, cell-free transcription/translation systems can be employed to produce polypeptides comprising an amino acid sequence or subsequence of, e.g., SEQ ID NO: 3 or SEQ ID NO:4, or encoded by the polynucleotide sequences of the invention. A number of suitable in vitro transcription and translation systems are commercially available. A general guide to in vitro transcription and translation protocols is found in Tymms (1995) In vitro Transcription and Translation Protocols: Methods in Molecular Biology Volume 37, Garland Publishing, NY.

In addition, the polypeptides, or subsequences thereof, e.g., subsequences comprising antigenic peptides, can be produced manually or by using an automated system, by direct peptide synthesis using solid-phase techniques (see, Stewart et al. (1969) Solid-Phase Peptide Synthesis, W H Freeman Co, San Francisco; Merrifield J (1963) J Am Chem Soc 85:2149-2154). Exemplary automated systems include the Applied Biosystems 431A Peptide Synthesizer (Perkin Elmer, Foster City, Calif.). If desired, subsequences can be chemically synthesized separately, and combined using chemical methods to provide full-length polypeptides.

Modified Amino Acids

Expressed polypeptides of the invention can contain one or more modified amino acid. The presence of modified amino acids can be advantageous in, for example, (a) increasing polypeptide serum half-life, (b) reducing polypeptide antigenicity, (c) increasing polypeptide storage stability, etc. Amino acid(s) are modified, for example, co-translationally or post-translationally during recombinant production (e.g., N-linked glycosylation at N-X-S/T motifs during expression in mammalian cells) or modified by synthetic means (e.g., via PEGylation).

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like, as well as amino acids modified by conjugation to, e.g., lipid moieties or other organic derivatizing agents. References adequate to guide one of skill in the modification of amino acids are replete throughout the literature. Example protocols are found in Walker (1998) Protein Protocols on CD-ROM Human Press, Towata, N.J.

Fusion Proteins

The present invention also provides fusion proteins comprising fusions of the sequences of the invention (e.g., encoding CGRP receptors) or fragments thereof with, e.g., immunoglobulins (or portions thereof), sequences encoding, e.g., GFP (green fluorescent protein), etc. Nucleotide sequence encoding such fusion proteins are another aspect of the invention. Fusion proteins of the invention are optionally used for, e.g., similar applications (including, e.g., therapeutic, prophylactic, diagnostic, experimental, etc. applications as described herein) as the non-fusion proteins of the invention. Optionally, the fusion proteins are used in elucidation of agonists/antagonists of CGRP receptors, etc. In addition to fusion with immunoglobulin sequences and marker sequences, the proteins of the invention are also optionally fused with, e.g., sequences which allow sorting of the fusion proteins and/or targeting of the fusion proteins to specific cell types, regions, etc.

Antibodies

The polypeptides of the invention can be used to produce antibodies specific for the polypeptide of, e.g., SEQ ID NO: 3 or SEQ ID NO:4 and/or polypeptides encoded by the polynucleotides of the invention, e.g., SEQ ID NO:1-SEQ ID NO:2, and conservative variants thereof. Antibodies specific for the above mentioned polypeptides are useful, e.g., for diagnostic and therapeutic purposes, e.g., related to the activity, distribution, and expression of target polypeptides. For example, antibodies that block receptor binding or that have either agonist or antagonist activity for the CGRP receptor, are useful for certain therapeutic applications. See, below for more information on therapeutic uses of the current invention.

Antibodies specific for the polypeptides of the invention can be generated by methods well known in the art. Such antibodies can include, but are not limited to, polyclonal, monoclonal, chimeric, humanized, single chain, Fab fragments and fragments produced by an Fab expression library.

Polypeptides do not require biological activity for antibody production (e.g., full length functional JPr-CGRP-R is not required). However, the polypeptide or oligopeptide must be antigenic. Peptides used to induce specific antibodies typically have an amino acid sequence of at least about 4 amino acids, and often at least 5 or 10 amino acids. Short stretches of a polypeptide can be fused with another protein, such as keyhole limpet hemocyanin, and antibody produced against the chimeric molecule.

Numerous methods for producing polyclonal and monoclonal antibodies are known to those of skill in the art, and can be adapted to produce antibodies specific for the polypeptides of the invention, e.g., SEQ ID NO: 3 or SEQ ID NO:4 and/or encoded by SEQ ID NO:1-SEQ ID NO:2, etc. See, e.g., Coligan (1991) Current Protocols in Immunology Wiley/Greene, NY; Paul (ed.) (1998) Fundamental Immunology, Fourth Edition, Lippincott-Raven, Lippincott Williams & Wilkins; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward, et al. (1989) Nature 341: 544-546. Specific monoclonal and polyclonal antibodies and antisera will usually bind with a K_{D} of, e.g., at least about 0.1 μM, at least about 0.01 μM or better, and, typically and at least about 0.001 μM or better.

For certain therapeutic applications, humanized antibodies are desirable. Detailed methods for preparation of chimeric (humanized) antibodies can be found in U.S. Pat. No. 5,482,856. Additional details on humanization and other antibody production and engineering techniques can be found in Borrebaeck (ed.) (1995) Antibody Engineering 2^{nd} Edition Freeman and Company, NY (Borrebaeck); McCafferty et al. (1996) Antibody Engineering, A Practical Approach IRL at Oxford Press, Oxford, England (McCafferty), and Paul (1995) Antibody Engineering Protocols Humana Press, Towata, N.J. (Paul). Additional details regarding specific procedures can be found, e.g., in Ostberg et al. (1983), Hybridoma 2: 361-367, Ostberg, U.S. Pat. No. 4,634,664, and Engelman et al., U.S. Pat. No. 4,634,666.

Administration in patients

In some aspects, the present invention provides for the administration of one or more of the nucleic acids herein, e.g., for gene therapy and/or for the administration of one or more protein herein as a prophylactic or therapeutic agent to a subject, including, e.g., a mammal, including, e.g., a human, primate, mouse, pig, cow, goat, rabbit, rat, guinea pig, hamster, horse, and/or sheep. In addition, modulators of expression of genes encoding the nucleic acids or proteins herein and/or activity modulators of the proteins herein can be administered to regulate, e.g., CGRP binding to CGRP-receptors (optionally in select tissues, etc.).

Whether the therapeutic agent is a nucleic acid, a protein or a modulator of an activity of a nucleic acid or protein, administration is by any of the routes normally used for introducing a molecule into ultimate contact with blood or tissue cells. Suitable methods of administering compositions in the context of the present invention to a patient are available, and, although more than one route can be used to administer a particular composition, a particular route can provide a more immediate and more effective reaction than another route.

The invention also includes compositions comprising any nucleic acid or any isolated or recombinant polypeptide described above and an excipient, e.g., a pharmaceutically acceptable excipient. Transgenic animals, which include any nucleic acid or polypeptide above, e.g., produced by introduction of a vector, are also a feature of the invention. Methods for treating (e.g., postmenopausal bone loss, vasodilation, migraines, chronic pain, diabetes, inflammation, cancer, obesity, Paget's disease, vomiting, benign prostatic hypertrophy, depression, psychosis, allergies, asthma, ulcers, angina pectoris, acute heart failure, hypotension, urinary retention, myocardial infarction, etc.) by administering to a patient an effective amount of at least one expression vector and/or an effective amount of at least one isolated or recombinant polypeptide described above are also included in the present invention.

Pharmaceutically acceptable excipients or carriers are determined in part by the particular composition being administered, as well as by the particular method used to administer the composition. Accordingly, there are a wide variety of suitable formulations of pharmaceutical compositions of the present invention.

Formulations suitable for parenteral administration, such as, for example, by intraarticular (in the joints), intravenous, intramuscular, intradermal, subdermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Parenteral administration and intravenous administration are one class of preferred methods of administration. Formulations can be presented in unit-dose or multi-dose sealed containers, such as ampules and vials.

Injection solutions and suspensions can be prepared from sterile powders, granules, and tablets. Cells transduced by expression vectors or gene therapy vectors (e.g., in the context of ex vivo gene therapy) can also be administered intravenously or parenterally as described above.

Formulations suitable for oral administration can consist of, e.g., liquid solutions, such as an effective amount of the packaged nucleic acid suspended in diluents, such as water, saline, buffered saline, ethanol, glycerol, dextrose, PEG 400 and combinations thereof; capsules, sachets or tablets, each containing a predetermined amount of the active ingredient, as liquids, solids, granules or gelatin; suspensions in an appropriate liquid; and/or suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient (e.g., a component of the invention) and a flavor or flavoring agent and usually sucrose and acacia or tragacanth. Additionally, pastilles comprising the active ingredient (e.g., a component of the invention) in an inert base, such as gelatin and glycerin or sucrose and acacia emulsions, gels, and the like containing, in addition to the active ingredient, carriers, etc., known to those of skill in the art are part of the invention.

The materials, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid or polypeptide with a suppository base. Suitable suppository bases include natural or synthetic triglycerides or paraffin hydrocarbons. In addition, it is also possible to use gelatin rectal capsules that consist of a combination of materials with a base, including, for example, liquid triglycerides, polyethylene glycols, and paraffin hydrocarbons.

The dose administered to a patient, in the context of the present invention should be sufficient to affect a beneficial prophylactic or therapeutic response in the patient over time. The dose will be determined by the efficacy of the particular composition employed and the condition of the patient, as well as the body weight or surface area of the patient to be treated. The size of the dose also will be determined by the existence, nature, and extent of any adverse side-effects that accompany the administration of a particular composition (e.g., gene therapy vector, transduced cell type, protein or activity modulator) in a particular patient.

In determining an effective amount to be administered in the treatment or prophylaxis of, e.g., postmenopausal bone loss, vasodilation, migraines, chronic pain, diabetes, inflammation, cancer, obesity, Paget's disease, vomiting, benign prostatic hypertrophy, depression, psychosis, allergies, asthma, ulcers, angina pectoris, acute heart failure, hypotension, urinary retention, myocardial infarction, etc., or an associated condition, a physician evaluates the progression or level of disease state/condition, vector toxicities, progression of disease, and, e.g., production of antibodies to the prophylactic/therapeutic composition, etc.

For example, in one aspect, the dose equivalent of a naked nucleic acid encoding a nucleic acid of the invention (e.g., SEQ ID NO: 1 or 2) herein is from about 0.1 μg to 1 mg for a typical 70 kilogram patient, and doses of vectors which include a gene therapy or expression vector, such as a retroviral particle, are calculated to yield an approximately equivalent amount of a nucleic acid.

In the practice of this invention, compositions can be administered, for example, by intravenous infusion, orally, topically, intraperitoneally, intravesically or intrathecally. The method of administration will often be local, oral, rectal or intravenous, but materials can also be applied in a suitable vehicle for the topical treatment of related conditions. The agents of this invention can supplement treatment of, e.g., postmenopausal bone loss, vasodilation, migraines, chronic pain, diabetes, inflammation, cancer, obesity, Paget's disease, vomiting, benign prostatic hypertrophy, depression, psychosis, allergies, asthma, ulcers, angina pectoris, acute heart failure, hypotension, urinary retention, myocardial infarction, etc., or related conditions by any known conventional therapy, including pain medications, biologic response modifiers and the like.

For administration, compositions of the present invention can be administered at a rate determined by the LD-50 of composition and the side-effects of the composition at various concentrations, as applied to the mass and overall health of the patient. Administration can be accomplished via single or divided doses.

For ex vivo therapy, transduced cells are prepared for reinfusion according to established methods. See, Abrahamsen et al. (1991) J Clin Apheresis 6:48-53; Carter et al. (1988) J Clin Arpheresis 4:113-117; Aebersold et al. (1988), J Immunol Methods 112: 1-7; Muul et al. (1987) J Immunol Methods 101:171-181 and Carter et al. (1987) Transfusion 27:362-365. As an illustration (but not as a limitation), e.g., after a period of about 2-4 weeks in culture, the cells should number between 1×10⁸ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the prophylactic/therapeutic agent (e.g., a CGRP receptor of the invention).

In one embodiment, in ex vivo methods, one or more cells, or a population of the subject's cells of interest, e.g., cells, tissue sample, blood cells, are obtained or removed from the subject and contacted with an amount of a CGRP receptor molecule of the invention, e.g., nucleic acids or subsequences thereof or isolated or recombinant polypeptides or subsequences thereof or antibodies, that is effective in prophylactically or therapeutically treating a condition. The contacted cells are then returned or delivered to the subject to the site from which they were obtained or to another site of interest in the subject to be treated. Contacted cells can also be grafted onto a tissue or system site of interest in the subject using standard and well-known grafting techniques or, e.g., delivered to the blood or lymph system using standard delivery or transfusion techniques. In another embodiment, a construct comprising a molecule, e.g., a nucleic acid sequence of the invention, e.g., SEQ ID NO: 1 or SEQ ID NO: 2, etc., that encodes a biologically active peptide that is effective in prophylactically or therapeutically treating a condition, is introduced into the one or more cells of interest or a population of cells of interest of the subject. A sufficient amount of the construct and a controlling promoter is used such that uptake of the construct (and promoter) into the cell(s) occurs and sufficient expression of the biologically active peptide (e.g., a CGRP receptor of the invention) produces an amount of the biologically active molecule effective to prophylactically or therapeutically treat the condition. Expression of the target nucleic acid can either be induced or occur naturally and a sufficient amount of the molecule is expressed and effective to treat the disease or condition at the site or tissue system.

In another embodiment, the invention provides in vivo methods in which one or more cells or a population of the subject's cells of interest are contacted directly or indirectly with an amount of a molecule(s) (polypeptides and/or polynucleotides of CGRP receptors of the invention) effective in prophylactically or therapeutically treating a condition. In direct contact/administration formats, the molecule(s) is typically administered or transferred directly to the cells to be treated or to the tissue site of interest (e.g., gastrointestinal tissue) by any of a variety of formats, which include injection, e.g., by a needle and/or syringe, vaccine, gene gun delivery, or pushing into gastrointestinal tissue. The molecule(s) can be delivered as described above, or placed within a cavity of the body (including, e.g., during surgery).

In in vivo indirect contact/administration formats, the molecule(s) is administered or transferred indirectly to the cells to be treated or to the tissue site of interest, such as, e.g., gastric tissue, nerve tissue, etc., lymphatic system, or blood cell system, etc, by contacting or administering the molecule(s) of the invention directly to one or more cells or population of cells from which treatment can be facilitated. For example, nerve cells within the body of the subject can be treated by contacting cells of the blood or lymphatic system with a sufficient amount of the molecule such that delivery of the molecule to the site of interest occurs and effective prophylactic or therapeutic treatment results. Such contact, administration, or transfer is typically made by using one or more of the routes or modes of administration described above.

In one embodiment, the invention provides in vivo methods. Typically, one or more cells of interest or a population of subject's cells are transformed in the body of the subject by contacting the cell(s) or population of cells with (or administering or transferring to the cell(s) or population of cells using one or more of the routes or modes of administration described above) a polynucleotide construct comprising a nucleic acid sequence of the invention that encodes a biologically active molecule of interest (e.g., a CGRP receptor polynucleotide of the invention) that is effective in prophylactically or therapeutically treating the condition, e.g., postmenopausal bone loss, vasodilation, migraines, chronic pain, diabetes, inflammation, cancer, obesity, Paget's disease, vomiting, benign prostatic hypertrophy, depression, psychosis, allergies, asthma, ulcers, angina pectoris, acute heart failure, hypotension, urinary retention, myocardial infarction, etc. Expression of the nucleic acid can be induced or occur naturally such that an amount of the encoded CGRP receptor polypeptide expressed is sufficient and effective to treat the condition or disease state. The polynucleotide construct can include a promoter sequence (e.g., CMV promoter sequence) and optionally, one or more additional nucleotide sequences of the invention, adjuvant, or co-stimulatory molecule, or other polypeptide of interest.

A variety of viral vectors suitable for in vivo transduction and expression in an organism are known. Such vectors include retroviral vectors (see, Miller (1992) Curr Top Microbiol Immunol 158:1-24; Salmons and Gunzburg (1993) Human Gene Therapy 4:129-141; Miller et al. (1994) Methods in Enzymology 217: 581-599), adeno-associated vectors (reviewed in Carter (1992) Curr Opinion Biotech 3: 533-539; Muzcyzka (1992) Curr Top Microbiol Immunol 158: 97-129) and other viral vectors (as generally described in, e.g., Jolly (1994) Cancer Gene Therapy 1:51-64; Latchman (1994) Molec Biotechnol 2:179-195; and Johanning et al. (1995) Nucl Acids Res 23:1495-1501).

In general, gene therapy provides methods for combating diseases, e.g., see, above, and some forms of congenital defects such as enzyme deficiencies. Various textbooks describe gene therapy protocols which can be used with the present invention by introducing nucleic acids, e.g., one or more of SEQ ID NO:1 or SEQ ID NO: 2, into patient. One example is Robbins (1996) Gene Therapy Protocols, Humana Press, NJ, and Joyner (1993) Gene Targeting: A Practical Approach, IRL Press, Oxford, England.

In addition to the references cited above, several approaches for introducing nucleic acids into cells in vivo, ex vivo and in vitro are also described below along with the references cited within. These include liposome based gene delivery (Debs and Zhu (1993) WO 93/24640 and U.S. Pat. No. 5,641,662; Mannino and Gould-Fogerite (1988) BioTechniques 6(7): 682-691; Rose, U.S. Pat No. 5,279,833; Brigham (1991) WO 91/06309; and Feigner et al. (1987) Proc Natl Acad Sci USA 84: 7413-7414); Brigham et al. (1989) Am J Med Sci, 298:278-281; Nabel et al. (1990) Science, 249:1285-1288; Hazinski et al. (1991) Am J Resp Cell Molec Biol, 4:206-209; and Wang and Huang (1987) Proc Natl Acad Sci USA, 84:7851-7855); adenoviral vector mediated gene delivery, e.g., to treat cancer (see, e.g., Chen et al. (1994) Proc Natl Acad Sci USA 91: 3054-3057; Tong et al. (1996) Gynecol Oncol 61: 175-179; Clayman et al. (1995) Cancer Res 5: 1-6; O'Malley et al. (1995) Cancer Res 55: 1080-1085; Hwang et al. (1995) Am J Respir Cell Mol Biol 13: 7-16; Haddada et al. (1995) Curr Top Microbiol Immunol 199 (Pt. 3): 297-306; Addison et al. (1995) Proc Natl Acad Sci USA 92: 8522-8526; Colak et al. (1995) Brain Res 691: 76-82; Crystal (1995) Science 270: 404-410; Elshami et al. (1996) Human Gene Ther 7: 141-148; Vincent et al. (1996) J Neurosurg 85: 648-654). Other delivery systems include replication-defective retroviral vectors harboring therapeutic polynucleotide sequence as part of the retroviral genome, particularly with regard to simple MuLV vectors (Miller et al. (1990) Mol Cell Biol 10:4239 (1990); Kolberg (1992) J NIH Res 4:43, and Cornetta et al. (1991) Hum Gene Ther 2:215), nucleic acid transport coupled to ligand-specific, cation-based transport systems (Wu and Wu (1988) J Biol Chem, 263:14621-14624) and naked DNA expression vectors (Nabel et al. (1990), supra); and Wolff et al. (1990) Science, 247:1465-1468). In general, these approaches can be adapted to the invention by incorporating nucleic acids, e.g., one or more of SEQ ID NO: 1 to SEQ ID NO: 2 herein, into the appropriate vectors.

In addition to expression of the nucleic acids of the invention as gene replacement nucleic acids, the nucleic acids are also useful for sense and anti-sense suppression of expression, e.g., to down-regulate expression of a nucleic acid of the invention, once expression of the nucleic acid is no-longer desired in the cell. Similarly, the nucleic acids of the invention, or subsequences or anti-sense sequences thereof, can also be used to block expression of naturally occurring homologous nucleic acids which encode a subject's innate CGRP-receptor expression, etc. A variety of sense and anti-sense technologies are known in the art, e.g., as set forth in Lichtenstein and Nellen (1997) Antisense Technology: A Practical Approach IRL Press at Oxford University, Oxford, England, and in Agrawal (1996) Antisense Therapeutics Humana Press, NJ, and the references cited therein.

Kits and Reagents

The present invention is optionally provided to a user as a kit. For example, a kit of the invention contains one or more nucleic acid, polypeptide, antibody, or cell line described herein (e.g., comprising, or with, a CGRP receptor of the invention). Most often, the kit contains a diagnostic nucleic acid or polypeptide, e.g., antibody, probe set, e.g., as a cDNA micro-array packaged in a suitable container, or other nucleic acid such as one or more expression vector. The kit typically further comprises, one or more additional reagents, e.g., substrates, labels, primers, for labeling expression products, tubes and/or other accessories, reagents for collecting samples, buffers, hybridization chambers, cover slips, etc. The kit optionally further comprises an instruction set or user manual detailing preferred methods of using the kit components for discovery or application of diagnostic gene sets, etc.

When used according to the instructions, the kit can be used, e.g., for evaluating expression or polymorphisms in a subject sample, i.e., for evaluating a disease state or condition, or for evaluating effects of a pharmaceutical agent or other treatment intervention on progression of a disease state or condition in a cell or organism.

In an additional aspect, the present invention provides system kits embodying the methods, composition, systems and apparatus herein. System kits of the invention optionally comprise one or more of the following: (1) an apparatus, system, system component or apparatus component; (2) instructions for practicing methods described herein, and/or for operating the apparatus or apparatus components herein and/or for using the compositions herein. In a further aspect, the present invention provides for the use of any apparatus, apparatus component, composition or kit herein, for the practice of any method or assay herein, and/or for the use of any apparatus or kit to practice any assay or method herein.

Additionally, the kits can include one or more translation system as noted above (e.g., a cell),one or more unnatural amino acid, e.g., with appropriate packaging material, containers for holding the components of the kit, instructional materials for practicing the methods herein and/or the like. Similarly, products of the translation systems (e.g., proteins such as CGRP receptors analogues comprising unnatural amino acids) can be provided in kit form, e.g., with containers for holding the components of the kit, instructional materials for practicing the methods herein and/or the like.

Screening

In some embodiments of the invention, the polypeptides (e.g., CGRP receptors) are optionally utilized in various screening procedures. The characterization of the JPr-CGRP-R and other polypeptides (and polynucleotides, etc.) of the invention allow for, e.g., the screening of/for, e.g., agonists and antagonists to CGRP-receptors. For example, libraries of, e.g., peptides, peptide analogues, etc. are readily available commercially and can be used, e.g., to screen to test the activity of receptors, to test for modulatory activity of the library components on CGRP-r, etc. For example, the CGRP receptors are optionally used to screen for possible agonists and/or antagonists. Such agonists/antagonists are optionally peptides or non-peptides and are optionally used in the development of prophylactic and/or therapeutic treatments for a number of medical conditions or disease states (see, above). Some embodiments of the invention comprise methods of performing screening of CGRP-receptor modulating compounds (optionally such screening comprises high throughput screening as explained below). Such methods of screening comprise interacting a putative CGRP-receptor modulating compound with a polypeptide (e.g., CGRP receptor) of the invention wherein the receptor comprises a polypeptide sequence that comprises at least 91%, at least 92%, at least 93%, at least 94%, at least 95%, at least 96%, at least 97%, at least 98%, or at least 99% or more identity to SEQ ID NO:3 or SEQ ID NO:4.

The receptor polypeptides of the current invention are involved in a number of different physiological pathways and conditions as well as numerous diseases and medical conditions. See, above. Therefore, it is useful to utilize the polypeptides of the current invention to devise screening methods to identify and/or characterize compounds that, e.g., stimulate or inhibit the function of the receptors of the invention. Thus, the present invention provides methods of screening compounds to identify ones which stimulate or inhibit the function of the receptor polypeptides of the invention either directly or indirectly (e.g., through direct binding with the CGRP-receptors ,or through competition with other modulators, or though binding to other modulators, etc.). The agonists and antagonists of the CGRP-receptors of the invention that are found can then optionally be used for therapeutic and/or prophylactic treatment for such diseases as mentioned above.

Molecules and compounds to be screened are optionally identified from a variety of sources, e.g., cells, cell-free preparations, chemical libraries, natural product mixtures, etc. (many examples of all of such are commercially available). The agonists/antagonists (i.e., CGRP-receptor modulatory molecules/compounds) are optionally natural or modified substrates, ligands, receptors, or enzymes of the receptors herein, or they optionally comprise, e.g., functional or structural mimetics of such. See, e.g., Coligan et al., Current Protocols in Immunology 1(2):Chapter 5 (1991). Modulatory molecules also optionally comprise, e.g., oligonucleotides, proteins (e.g., including ones that are closely related to the ligands, substrates, etc. of the CGRP-receptors of the invention such as CGRP), and fragments of such proteins. Additionally, small molecules (e.g., non-polypeptides) that optionally bind to, e.g., another modulator of the CGRP-receptor, thus, preventing that modulator from its customary action, etc. are also possible. Additionally, through examination of the structure of, e.g., CGRP and of the CGRP-receptors of the invention, additional putative modulators are optionally derived/constructed based upon the 3-dimensional structure of the CGRP (or other molecule known to bind to the CGRP-receptors of the invention), thus creating and testing other possible agonists/antagonists for use with the CGRP-receptors of the invention. For example, a particular antagonist of a CGRP-receptor of the invention may thus be examined and modified in order to produce, e.g., an antagonist with a longer serum half-life, or with a stronger binding constant. In turn, the improved antagonist may be used in the therapeutic and/or prophylactic treatments of a disease state or condition involving that CGRP-receptor.

The screenings comprising the current invention can optionally take numerous forms. For example, the methods may simply measure the binding of a putative agonist/antagonist to the CGRP-receptor. Alternatively, the methods can measure the binding of such putative agonist/antagonist to, e.g., cells, membranes, matrices, etc., bearing the CGRP-receptors (or, e.g., fusion proteins and/or fragments comprising the CGRP receptors). Such measurement can be, e.g., via measure of a label that is directly or indirectly associated with the putative modulator. In other embodiments, screening may involve competition with or between labeled competitors.

The screenings of the invention can test, e.g., if a putative modulator generates a signal by activation or inhibition of the receptors of the invention through detection systems appropriate to the cells/systems comprising the receptors. For example, inhibitors of activation are typically tested in the presence of a known agonist and the effect on activation by the agonist by the presence of the putative modulator is measured. Constitutively active receptors are optionally employed in screening methods using inverse agonists or inhibitors, or in the absence of an agonist or inhibitor, by testing whether the putative modulator results in inhibition of activation of the receptor. The screening methods of the invention can also comprise the steps of mixing a putative modulator with a solution containing a polypeptide of the invention, thus forming a mixture, and measuring activity of a CGRP-receptor in such mixture as compared to a control. Other screenings optionally comprise fusion proteins comprising a CGRP-receptor of the invention (or a fragment thereof) and an immunoglobulin. See, above.

One optional method of screening in the current invention tests for the modulatory effect of a putative agonist/antagonist by measuring cAMP and/or adenylate cyclase stimulation/concentration/accumulation. See, e.g., Example 1 below. In such embodiments, a cell (typically a eukaryotic cell) is transfected with a CGRP-receptor of the invention in such a manner that the receptor is properly expressed and situated (e.g, on the cell surface). The putative modulators are then interacted with the cell comprising the CGRP-receptors of the invention and the level, etc. of cAMP and/or adenylate cyclase is measured (e.g., intracellularly). For example, if an antagonist binds to the CGRP-receptor then the receptor will not be properly stimulated (e.g., as it would if a non-antagonist were to bind to it), thus leading to changed levels of receptor-mediated cAMP and/or adenylate cyclase.

In other screenings herein, the CGRP-receptors of the invention are optionally used to detect or characterize antagonist/agonists which are then, in turn, used to identify other membrane bound (or soluble) receptors of similar type to those of the invention. Receptor binding assays, etc., that are well known to those of skill in the art are optionally used in such situations. For example, ligand binding-crosslinking with, e.g., radiolabeled modulators, fluorescently tagged modulators, or other such marked modulators can be used in such embodiments. See, Example 1, below. Additionally, techniques such as surface plasmon resonance and spectroscopy are optionally used to identify agonists/antagonists that, e.g., compete with each other or with other possible ligands to the CGRP-receptors of the invention. Numerous receptor binding assays are well known to those in the art and can be easily modified to be utilized in the current methods. Also, plaques or cells constituting libraries can also be screened directly for production of e.g., proteins, either by detecting hybridization, protein activity, protein binding to antibodies, or the like, e.g., for production of agonists/antagonists of the CGRP receptor.

In other embodiments of the screenings of the invention, polynucleotides of the invention are optionally used to detect complementary versions of themselves (e.g., within a subject, etc.). Such screenings, for example, are optionally used as diagnostic tools to detect, e.g., a malfunctioned or dysfunctional polynucleotide in a subject which might lead to a disease state or condition due to overexpression or underexpression of the polynucleotide and, thus, of the polypeptide. Such screenings are also optionally used to determine subjects who are carriers for disease states or conditions characterized by such dysfunctional polypeptides, but who do not display the disease state/condition. A subject's nucleic acid is optionally obtained via any number of methods that are well known to those in the art (e.g., blood, saliva, cells, tissue biopsy, etc.) and is then examined/characterized. For example, the dysfunctional polypeptides are optionally screened through complementary binding with the polynucleotides of the invention, and/or followed by, e.g., sequencing, gel mobility, hybridization studies, chemical footprinting, etc.

In yet other embodiments of the invention, polypeptides of the invention are optionally used (directly or indirectly) as, e.g., diagnostic tools, etc. For example, as explained herein, the polypeptides of the invention are optionally used to develop specific antibodies. Such antibodies are optionally used to, e.g., characterize a subject's endogenous CGRP-receptors, measure levels or presence of endogenous CGRP-receptors in a subject, etc. Additionally, screenings involving the polypeptides of the invention can optionally comprise, e.g., competitive binding assays, and the like to, e.g., determine levels/characterization of CGRP-receptor ligands (e.g., CGRP or a modulatory molecule) in a subject. In other words, the polypeptides of the invention (either as cell surface molecules, as matrix bound molecules, or as free molecules which are optionally soluble fusion proteins) can be used to bind a subject's ligand (e.g., CGRP) or other modulatory molecule, thus measuring/characterizing such molecule. As explained throughout, CGRP-receptors are suspected or known to be involved in a number of disease states and conditions. Thus, this method is also optionally utilized as a therapeutic/prophylactic treatment (e.g., to bind up excess ligand or modulatory molecules, etc.) as well as a diagnostic/research tool.

Some screenings of the invention comprise high throughput assays, wherein it is possible to screen up to several thousand different molecules in a single day. For example, each well of a microtiter plate can be used to run a separate assay, or, if concentration or incubation time effects are to be observed, every 5-10 wells can test a single agonists/antagonists (e.g., at different concentrations and/or under different conditions). Thus, a single standard microtiter plate can assay about 100 (e.g., 96) reactions. If 1536 well plates are used, then a single plate can easily assay from about 100 to about 1500 different reactions. It is possible to assay several different plates per day; assay screens for up to about 6,000-20,000 different assays (e.g., involving different nucleic acids, encoded proteins, concentrations, putative agonists or antagonists, etc.) are possible using the integrated systems of the invention. More recently, microfluidic approaches to reagent manipulation have been developed, e.g., by Caliper Technologies (Mountain View, Calif.) which can provide very high throughput microfluidic assay methods.

Additionally, a number of well known robotic systems have also been developed for solution phase chemistries useful in assay systems. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a scientist. Any of the above devices are suitable for use with the present invention, e.g., for high-throughput screening of possible agonist/antagonist molecules for the CGRP receptors of the invention, etc. The nature and implementation of modifications to these devices (if any) so that they can operate as discussed herein with reference to the integrated system will be apparent to persons skilled in the relevant art.

High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like. Microfluidic approaches to reagent manipulation have also been developed, e.g., by Caliper Technologies (Mountain View, Calif.).

A variety of commercially available peripheral equipment and software is available for digitizing, storing and analyzing a digitized video or digitized optical or other assay images involved in the screenings herein, e.g., using PC (Intel x86 or pentium chip-compatible DOS™, OS2™ WINDOWS™, WINDOWS NT™, WINDOWS95™ or WINDOWS2000™ based machines), MACINTOSH™, UNIX based (e.g., SUN™ work station) computers or other similar systems. See, below for more discussion of possible computer systems involved. Additionally, current art computational hardware resources are fully adequate for practical use in the current invention, e.g., involved in the screening method of the current invention (any mid-range priced Unix system (e.g., for Sun Microsystems) or even higher end Macintosh or PCs will suffice). Current art in software technology is adequate (i.e., there are a multitude of mature programming languages and source code suppliers) for design of an upgradable open-architecture object-oriented genetic algorithm package, specialized for users with biological backgrounds.

As will be appreciated, the illustrations of various screenings, etc. herein should not be taken as limiting. Thus, those of skill in the art will understand that numerous other screening methods, formats, etc. can be applicable for use with the polynucleotides/polypeptides of the invention. The screenings herein, as well as other possible screenings, typically are capable of optimization, etc. depending upon, e.g., specific reaction conditions, parameters involved in the particular screenings, etc.

Digital Systems

The present invention provides digital systems, e.g., computers, computer readable media and integrated systems comprising character strings corresponding to the sequence information herein for the nucleic acids and isolated or recombinant polypeptides herein, including, e.g., those sequences listed herein and the various silent substitutions and conservative substitutions thereof. Integrated systems can further include, e.g., gene synthesis equipment for making genes corresponding to the character strings.

Various methods known in the art can be used to detect homology or similarity between different character strings, or can be used to perform other desirable functions such as to control output files, provide the basis for making presentations of information including the sequences and the like. Examples include BLAST, discussed infra. Computer systems of the invention can include such programs, e.g., in conjunction with one or more data file or data base comprising a sequence as noted herein.

Thus, different types of homology and similarity of various stringency and length between various CGRP receptors or fragments, etc. can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among 4 principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). See, below.

Thus, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™, Paradox™, GeneWorks™, or MacVector™ or other similar programs) can be adapted to the present invention by inputting a character string corresponding to one or more polynucleotides and polypeptides of the invention (either nucleic acids or proteins, or both). For example, a system of the invention can include the foregoing software having the appropriate character string information, e.g., used in conjunction with a user interface (e.g., a GUI in a standard operating system such as a Windows, Macintosh or LINUX system) to manipulate strings of characters corresponding to the sequences herein. As noted, specialized alignment programs such as BLAST can also be incorporated into the systems of the invention for alignment of nucleic acids or proteins (or corresponding character strings).

Systems in the present invention typically include a digital computer with data sets entered into the software system comprising any of the sequences herein. The computer can be, e.g., a PC (Intel x86 or Pentium chip- compatible DOS™, OS2™ WINDOWS™ WINDOWSNT™, WINDOWS95™, WINDOWS2000™, WINDOWS98™, LINUX based machine, a MACINTOSH™, Power PC, or a UNIX based (e.g., SUN™ work station) machine) or other commercially common computer that is known to one of skill. Software for aligning or otherwise manipulating sequences is available, or can easily be constructed by one of skill using a standard programming language such as Visualbasic, PERL, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation, e.g., of appropriate mechanisms or transport controllers to carry out the desired operation. The software can also include output elements for controlling nucleic acid synthesis (e.g., based upon a sequence or an alignment of sequences herein), comparisons of samples for differential gene expression, or other operations.

Nucleic acid and polypeptide sequence variants

As described herein, the invention provides for nucleic acid polynucleotide sequences and polypeptide amino acid sequences, e.g., CGRP-receptor sequences, and, e.g., compositions and methods comprising said sequences. Examples of said sequences, e.g., of CGRP receptors are disclosed herein. However, one of skill in the art will appreciate that the invention is not limited to those sequences disclosed herein and that the present invention also provides many related and unrelated sequences with the functions described herein, e.g., encoding a CGRP receptor.

One of skill will also appreciate that many variants of the disclosed sequences are included in the invention. For example, conservative variations of the disclosed sequences that yield a functionally identical sequence are included in the invention. Variants of the nucleic acid polynucleotide sequences, wherein the variants hybridize to at least one disclosed sequence, are considered to be included in the invention. Unique subsequences of the sequences disclosed herein, as determined by, e.g., standard sequence comparison techniques, are also included in the invention.

Conservative variations

Owing to the degeneracy of the genetic code, “silent substitutions” (i.e., substitutions in a nucleic acid sequence which do not result in an alteration in an encoded polypeptide) are an implied feature of every nucleic acid sequence of the invention which encodes an amino acid. Similarly, “conservative amino acid substitutions,” in one or a few amino acids in an amino acid sequence are substituted with different amino acids with highly similar properties, are also readily identified as being highly similar to a disclosed construct such as those herein. Such conservative variations of each disclosed sequence are a feature of the present invention.

“Conservative variations” of a particular nucleic acid sequence refers to those nucleic acids which encode identical or essentially identical amino acid sequences, or, where the nucleic acid does not encode an amino acid sequence, to essentially identical sequences, see, Table 1 below. One of skill will recognize that individual substitutions, deletions or additions which alter, add or delete a single amino acid or a small percentage of amino acids (typically less than 5%, more typically less than 4%, 3%, 2% or 1%) in an encoded sequence are “conservatively modified variations” where the alterations result in the deletion of an amino acid, addition of an amino acid, or substitution of an amino acid with a chemically similar amino acid. Thus, “conservative variations” of a listed polypeptide sequence of the present invention include substitutions of a small percentage, typically less than 5%, more typically less than 4%, 3%, 2% or 1%, of the amino acids of the polypeptide sequence, with a conservatively selected amino acid of the same conservative substitution group. Finally, the addition of sequences which do not alter the encoded activity of a nucleic acid molecule, such as the addition of a non-functional sequence, is a conservative variation of the basic nucleic acid.

For example, if 2 conservative substitutions were localized in the region corresponding to amino acids 1-10 of SEQ ID NO:4 (i.e., MDL HLF DYAE), examples of such conservatively substituted variations include, e.g., MDI HLY DYAE (SEQ ID NO:5), and MDL HLF EWAE (SEQ ID NO:6) and the like, in accordance with the conservative substitutions listed in Table 1 below. In this example, conservative substitutions are underlined.

TABLE 1
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Nucleic Acid Hybridization

Comparative hybridization can be used to identify nucleic acids of the invention, including conservative variations of nucleic acids of the invention. This comparative hybridization method is a preferred method of distinguishing nucleic acids of the invention. In addition, target nucleic acids which hybridize to the nucleic acids represented by, e.g., SEQ ID NO:1 or SEQ ID NO:2, etc. under high, ultra-high and ultra- ultra-high stringency conditions are a feature of the invention. Examples of such nucleic acids include those with one or a few silent or conservative nucleic acid substitutions as compared to a given nucleic acid sequence.

A test target nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 5×-10× as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel, infra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions comprises a 0.2× SSC wash at 65° C. for 15 minutes (see, Sambrook, infra for a description of SSC buffer). Often the high stringency wash is preceded by a low stringency wash to remove background probe signal. An example low stringency wash is 2× SSC at 40° C. for 15 minutes. In general, a signal to noise ratio of 5× (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993), supra, and in Hames and Higgins, 1 and 2. Stringent hybridization and wash conditions can easily be determined empirically for any test nucleic acid. For example, in determining highly stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tₘ for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

Unique subsequences

In one aspect, the invention provides a nucleic acid which comprises a unique subsequence in a nucleic acid selected from the sequence of CGRP receptors disclosed herein, e.g., SEQ ID NO:1, SEQ ID NO:2, etc. The unique subsequence is unique as compared to a nucleic acid corresponding to the nucleic acid corresponding to GenBank accession number X14048. Alignment can be performed using, e.g., BLAST set to default parameters. Any unique subsequence is useful, e.g., as a probe to identify the nucleic acids of the invention.

Similarly, the invention includes a polypeptide which comprises a unique subsequence in a polypeptide selected from the sequence of CGRP receptor disclosed herein, e.g., SEQ ID NO:3, SEQ ID NO:4, etc. Here, the unique subsequence is unique as compared to a polypeptide corresponding to, e.g., the amino acid corresponding to the polynucleotide sequence of GenBank accession number X14048.

The invention also provides for target nucleic acids which hybridize under stringent conditions to a unique coding oligonucleotide which encodes a unique subsequence in a polypeptide selected from the sequences of CGRP receptors of the invention wherein the unique subsequence is unique as compared to a polypeptide corresponding to any of the control polypeptides (sequences of, e.g., the nucleic acid corresponding to GenBank accession number X14048, see, below). Unique sequences are determined as noted above.

Sequence comparison, identity, and homology

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding a CGRP receptor, or the amino acid sequence of a CGRP receptor) refers to two or more sequences or subsequences that have at least about 90%, preferably 91%, most preferably 92%, 93%, 94%, 95%, 96%, 97%, 98%, 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, “substantial identity” exists over a region of the amino acid sequences that is at least about 50 residues in length, more preferably over a region of at least about 75 residues, and most preferably the sequences are substantially identical over at least about 100 residues, 150 residues, 200 residues, 250 residues, 275 residues, 300 residues, 325 residues, 350 residues, 355 residues, 360 residues, 361 residues, or 362 residues, or over the full length of the two sequences to be compared.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, Proc Natl Acad Sci USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J Mol Biol 215:403-410 (1990). Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (see, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff (1989) Proc Natl Acad Sci USA 89:10915).

In addition to calculating percent sequence identity, the BLAST algorithm also performs a statistical analysis of the similarity between two sequences (see, e.g., Karlin & Altschul, Proc Natl Acad Sci USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Defining Polypeptides by Immunoreactivity

Because the polypeptides of the invention provide a variety of new polypeptide sequences (e.g., comprising, CGRP receptors), the polypeptides also provide new structural features which can be recognized, e.g., in immunological assays. The generation of antisera which specifically bind the polypeptides of the invention, as well as the polypeptides which are bound by such antisera, are a feature of the invention.

For example, the invention includes polypeptides (e.g., CGRP receptor proteins) that specifically bind to or that are specifically immunoreactive with an antibody or antisera generated against an immunogen comprising an amino acid sequence selected from one or more SEQ ID NO:3 or SEQ ID NO:4, etc. To eliminate cross-reactivity with other homologues, the antibody or antisera is subtracted with the dog CGRP receptor (GenBank accession number X14048), e.g., the “control” polypeptide(s). Where the other control sequence (e.g., the canine CGRP receptor) corresponds to a nucleic acid, a polypeptide encoded by the nucleic acid is generated and used for antibody/antisera subtraction purposes.

In one typical format, the immunoassay uses a polyclonal antiserum which was raised against one or more polypeptide comprising one or more of the sequences corresponding to SEQ ID NO:3 or 4, etc. or a substantial subsequence thereof (i.e., at least about 30% of the full length sequence provided). The set of potential polypeptide immunogens derived from SEQ ID NO:3 or 4 are collectively referred to below as “the immunogenic polypeptides.” The resulting antisera is optionally selected to have low cross-reactivity against the control CGRP receptor homologues and any such cross-reactivity is removed, e.g., by immunoabsorbtion, with one or more of the control CGRP receptor homologues, prior to use of the polyclonal antiserum in the immunoassay.

In order to produce antisera for use in an immunoassay, one or more of the immunogenic polypeptides is produced and purified as described herein. For example, recombinant protein can be produced in a recombinant cell. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, e.g., Harlow and Lane (1988) Antibodies, A Laboratory Manual, Cold Spring Harbor Publications, New York, for a standard description of antibody generation, immunoassay formats and conditions that can be used to determine specific immunoreactivity). Additional references and discussion of antibodies is also found herein and can be applied here to defining polypeptides by immunoreactivity. Alternatively, one or more synthetic or recombinant polypeptide derived from the sequences disclosed herein is conjugated to a carrier protein and used as an immunogen.

Polyclonal sera are collected and titered against the immunogenic polypeptide in an immunoassay, for example, a solid phase immunoassay with one or more of the immunogenic proteins immobilized on a solid support. Polyclonal antisera with a titer of 10⁶ or greater are selected, pooled and subtracted with the control CGRP receptor polypeptide(s) to produce subtracted pooled titered polyclonal antisera.

The subtracted pooled titered polyclonal antisera are tested for cross reactivity against the control homologue(s) in a comparative immunoassay. In this comparative assay, discriminatory binding conditions are determined for the subtracted titered polyclonal antisera which result in at least about a 5-10 fold higher signal to noise ratio for binding of the titered polyclonal antisera to the immunogenic polypeptides as compared to binding to the control homologues. That is, the stringency of the binding reaction is adjusted by the addition of non-specific competitors such as albumin or non-fat dry milk, and/or by adjusting salt conditions, temperature, and/or the like. These binding conditions are used in subsequent assays for determining whether a test polypeptide (a polypeptide being compared to the immunogenic polypeptides and/or the control polypeptides) is specifically bound by the pooled subtracted polyclonal antisera. In particular, test polypeptides which show at least a 2-5× higher signal to noise ratio than the control receptor homologues under discriminatory binding conditions, and at least about a ½ signal to noise ratio as compared to the immunogenic polypeptide(s), shares substantial structural similarity with the immunogenic polypeptide as compared to the known receptor, etc., and is, therefore a polypeptide of the invention.

In another example, immunoassays in the competitive binding format are used for detection of a test polypeptide. For example, as noted, cross-reacting antibodies are removed from the pooled antisera mixture by immunoabsorbtion with the control polypeptides. The immunogenic polypeptide(s) are then immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control protein(s) to compete for binding to the pooled subtracted antisera is optionally determined as compared to the ability of the immunogenic polypeptide(s) to compete for binding to the antisera. Again, the percent cross-reactivity for the control polypeptide(s) is calculated, using standard calculations. Where the percent cross-reactivity is at least 5-10× as high for the test polypeptides as compared to the control polypeptide(s) and or where the binding of the test polypeptides is approximately in the range of the binding of the immunogenic polypeptides, the test polypeptides are said to specifically bind the pooled subtracted antisera.

In general, the immunoabsorbed and pooled antisera can be used in a competitive binding immunoassay as described herein to compare any test polypeptide to the immunogenic and/or control polypeptide(s). In order to make this comparison, the immunogenic, test and control polypeptides are each assayed at a wide range of concentrations and the amount of each polypeptide required to inhibit 50% of the binding of the subtracted antisera to, e.g., an immobilized control, test or immunogenic protein is determined using standard techniques. If the amount of the test polypeptide required for binding in the competitive assay is less than twice the amount of the immunogenic polypeptide that is required, then the test polypeptide is said to specifically bind to an antibody generated to the immunogenic protein, provided the amount is at least about 5-10× as high as for the control polypeptide.

As an additional determination of specificity, the pooled antisera is optionally fully immunosorbed with the immunogenic polypeptide(s) (rather than the control polypeptide(s)) until little or no binding of the resulting immunogenic polypeptide subtracted pooled antisera to the immunogenic polypeptide(s) used in the immunosorbtion is detectable. This fully immunosorbed antisera is then tested for reactivity with the test polypeptide. If little or no reactivity is observed (i.e., no more than 2× the signal to noise ratio observed for binding of the fully immunosorbed antisera to the immunogenic polypeptide), then the test polypeptide is specifically bound by the antisera elicited by the immunogenic protein.

Cloning, Mutagenesis and Expression of Biomolecules of Interest

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). These texts describe mutagenesis, the use of vectors, promoters and many other relevant topics related to, e.g., the generation of CGRP receptors, etc.

Various types of mutagenesis are optionally used in the present invention, e.g., to produce and/or isolate novel CGRP receptors and/or to further modify/mutate the polypeptides (e.g., CGRP receptors) of the invention. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like.

The above texts and examples found herein describe these procedures as well as the following publications (and references cited within): Sieber, et al., Nature Biotechnology, 19:456-460 (2001); Ling et al., Approaches to DNA mutagenesis: an overview, Anal Biochem 254(2): 157-178 (1997); Dale et al., Oligonucleotide-directed random mutagenesis using the phosphorothioate method, Methods Mol Biol 57:369-374 (1996); I. A. Lorimer, I. Pastan, Nucleic Acids Res 23, 3067-8 (1995); W. P. C. Stemmer, Nature 370, 389-91 (1994); Arnold, Protein engineering for unusual environments, Current Opinion in Biotechnology 4:450-455 (1993); Bass et al., Mutant Trp repressors with new DNA-binding specificities, Science 242:240-245 (1988); Fritz et al., Oligonucleotide-directed construction of mutations: a gapped duplex DNA procedure without enzymatic reactions in vitro, Nucl Acids Res 16: 6987-6999 (1988); Kramer et al., Improved enzymatic in vitro reactions in the gapped duplex DNA approach to oligonucleotide-directed construction of mutations, Nucl Acids Res 16: 7207 (1988); Sakamar and Khorana, Total synthesis and expression of a gene for the a-subunit of bovine rod outer segment guanine nucleotide-binding protein (transducin), Nucl Acids Res 14: 6361-6372 (1988); Sayers et al., Y-T Exonucleases in phosphorothioate-based oligonucleotide-directed mutagenesis, Nucl Acids Res 16:791-802 (1988); Sayers et al., Strand specific cleavage ofphosphorothioate-containing DNA by reaction with restriction endonucleases in the presence of ethidium bromide, (1988) Nucl Acids Res 16: 803-814; Carter, Improved oligonucleotide-directed mutagenesis using M13 vectors, Methods in Enzymol 154: 382-403 (1987); Kramer & Fritz Oligonucleotide-directed construction of mutations via gapped duplex DNA, Methods in Enzymol 154:350-367 (1987); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel et al., Rapid and efficient site-specific mutagenesis without phenotypic selection, Methods in Enzymol 154, 367-382 (1987); Zoller & Smith, Oligonucleotide-directed mutagenesis: a simple method using two oligonucleotide primers and a single-stranded DNA template, Methods in Enzymol 154:329-350 (1987); Carter, Site-directed mutagenesis, Biochem J 237:1-7 (1986); Eghtedarzadeh & Henikoff, Use of oligonucleotides to generate large deletions, Nucl Acids Res 14: 5115 (1986); Mandecki, Oligonucleotide-directed double-strand break repair in plasmids of Escherichia coli: a method for site-specific mutagenesis, Proc Natl Acad Sci USA, 83:7177-7181 (1986); Nakamaye & Eckstein, Inhibition of restriction endonuclease Nci I cleavage by phosphorothioate groups and its application to oligonucleotide-directed mutagenesis, Nucl Acids Res 14: 9679-9698 (1986); Wells et al., Importance of hydrogen-bond formation in stabilizing the transition state of subtilisin, Phil Trans R Soc Lond A 317: 415-423 (1986); Botstein & Shortle, Strategies and applications of in vitro mutagenesis, Science 229:1193-1201(1985); Carter et al., Improved oligonucleotide site-directed mutagenesis using M13 vectors, Nucl Acids Res 13: 4431-4443 (1985); Grundström et al., Oligonucleotide-directed mutagenesis by microscale ‘shot-gun’ gene synthesis, Nucl Acids Res 13: 3305-3316 (1985); Kunkel, Rapid and efficient site-specific mutagenesis without phenotypic selection, Proc Natl Acad Sci USA 82:488-492 (1985); Smith, In vitro mutagenesis, Ann Rev Genet 19:423-462(1985); Taylor et al., The use of phosphorothioate-modified DNA in restriction enzyme reactions to prepare nicked DNA, Nucl Acids Res 13: 8749-8764 (1985); Taylor et al., The rapid generation of oligonucleotide-directed mutations at high frequency using phosphorothioate-modified DNA, Nucl Acids Res 13: 8765-8787 (1985); Wells et al., Cassette mutagenesis: an efficient method for generation of multiple mutations at defined sites, Gene 34:315-323 (1985); Kramer et al., The gapped duplex DNA approach to oligonucleotide-directed mutation construction, Nucl Acids Res 12: 9441-9456 (1984); Kramer et al., Point Mismatch Repair, Cell 38:879-887 (1984); Nambiar et al., Total synthesis and cloning of a gene coding for the ribonuclease S protein, Science 223: 1299-1301 (1984); Zoller & Smith, Oligonucleotide-directed mutagenesis of DNA fragments cloned into M13 vectors, Methods in Enzymol 100:468-500 (1983); and Zoller & Smith, Oligonucleotide-directed mutagenesis using M13-derived vectors: an efficient and general procedure for the production of point mutations in any DNA fragment, Nucl Acids Res 10:6487-6500 (1982). Additional details on many of the above methods can be found in Methods in Enzymol Volume 154, which also describes useful controls for trouble-shooting problems with various mutagenesis, gene isolation, expression, and other methods.

Oligonucleotides, e.g., for use in mutagenesis of the present invention, e.g., mutating libraries of the CGRP receptors of the invention, or altering such, are typically synthesized chemically according to the solid phase phosphoramidite triester method described by Beaucage and Caruthers, Tetrahedron Letts 22(20):1859-1862, (1981) e.g., using an automated synthesizer, as described in Needham-VanDevanter et al., Nucleic Acids Res, 12:6159-6168 (1984).

In addition, essentially any nucleic acid can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (available at pkim@ccnet.com), HTI Bio-products, Inc. (www.htibio.com), BMA Biomedicals Ltd. (U.K.), Bio.Synthesis, Inc., and many others.

The present invention also relates to host cells and organisms comprising a CGRP receptor or other polypeptide and/or nucleic acid of the invention. Host cells are genetically engineered (e.g., transformed, transduced or transfected) with the vectors of this invention, which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation (see, From et al., Proc Natl Acad Sci USA 82, 5824 (1985), infection by viral vectors, high velocity ballistic penetration by small particles with the nucleic acid either within the matrix of small beads or particles, or on the surface (Klein et al., Nature 327, 70-73 (1987)). Berger, Sambrook, and Ausubel provide a variety of appropriate transformation methods.

The engineered host cells can be cultured in conventional nutrient media modified as appropriate for such activities as, for example, screening steps, activating promoters or selecting transformants. These cells can optionally be cultured into transgenic organisms.

Other useful references, e.g. for cell isolation and culture (e.g., for subsequent nucleic acid isolation) include Freshney (1994) Culture of Animal Cells, a Manual of Basic Technique, third edition, Wiley- Liss, New York and the references cited therein; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla.

Several well-known methods of introducing target nucleic acids into bacterial cells are available, any of which can be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria, (see, e.g., EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr Purif 6435: 10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Gherna et al. (eds.) published by the ATCC. Additional basic procedures for sequencing, cloning and other aspects of molecular biology and underlying theoretical considerations are also found in Watson et al. (1992) Recombinant DNA Second Edition Scientific American Books, NY.

### EXAMPLE

The following example is offered to illustrate, but not to limit the claimed invention.

### Example 1

### Production and Characterization of rat r-CGRP-R

Materials: ¹²⁵I-CGRP (50 mCi/mmol) was purchased from DuPont NEN (Boston, Mass.). ³H-Adenine was purchased from Amersham Life Sciences (Arlington Heights, Ill.). Dulbecco's Modified Eagle Medium (DMEM), Calf Serum, penicillin G, and streptomycin sulfate were purchased from Gibco-BRL (Gaithersburg, Md.). Bovine Serum Albumin was obtained from ICN Biomedicals, Inc. (Aurora, Ohio).

cDNA Cloning from a rat vas deferens cDNA library: A rat cDNA library was constructed from rat vas deferens via high and low stringency hybridization using ³²P labeled dog (RDC) cDNA probe. Of 64 hybridizing transcripts identified by screening, 4 candidate full-length transcripts were isolated and sequenced. The deduced sequence (JP-CGRP-R) comprises a >90% sequence homology with the dog RDC-1. However, very little sequence similarity exists between the JP-CGRP receptor and prior cloned human CGRP receptors of the subtype represented by, e.g., Skb-CGRP-R.

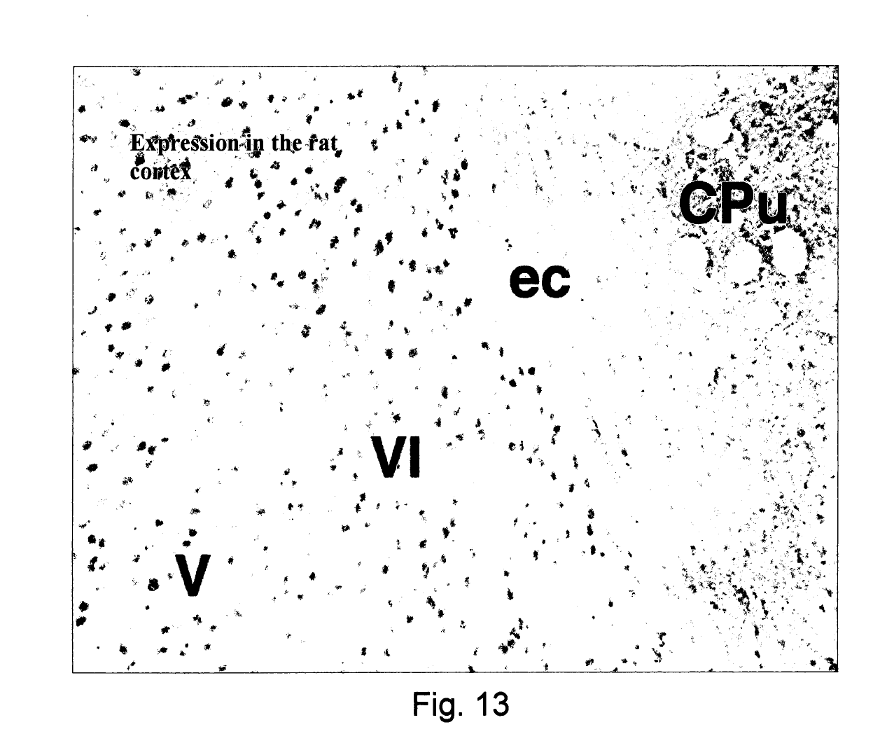

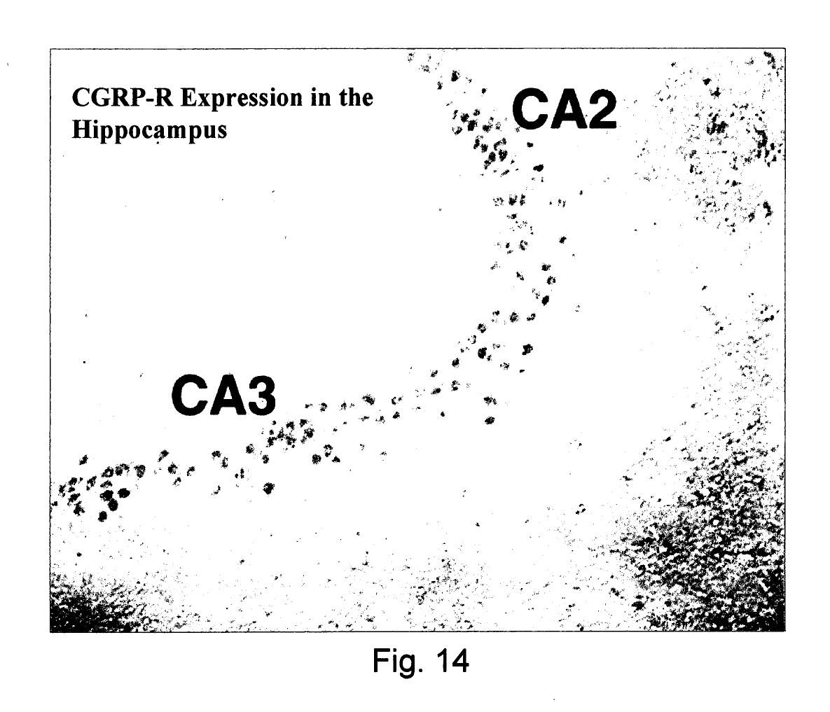

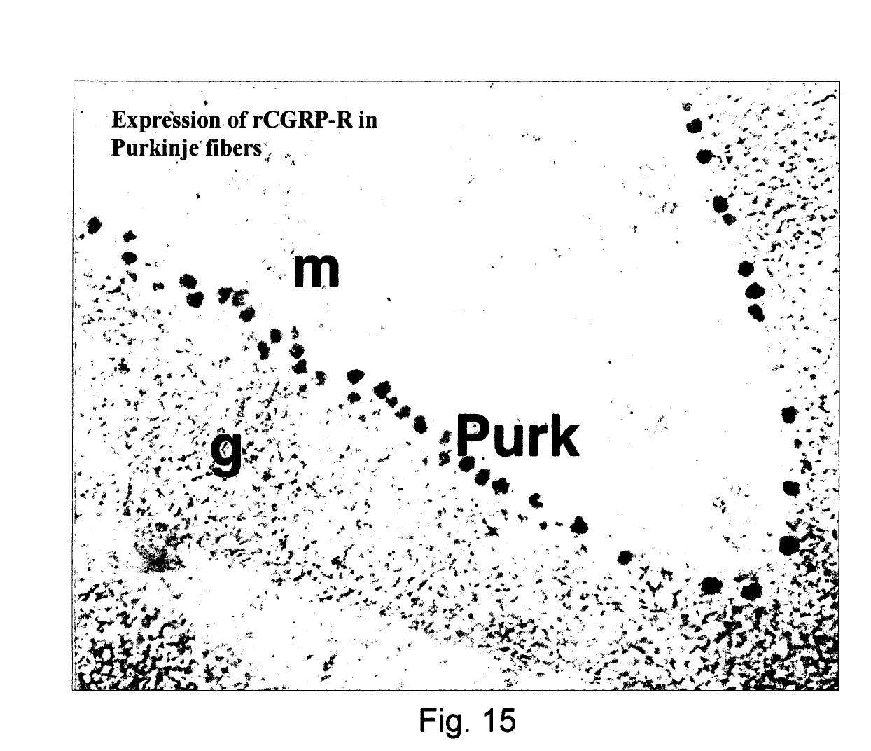

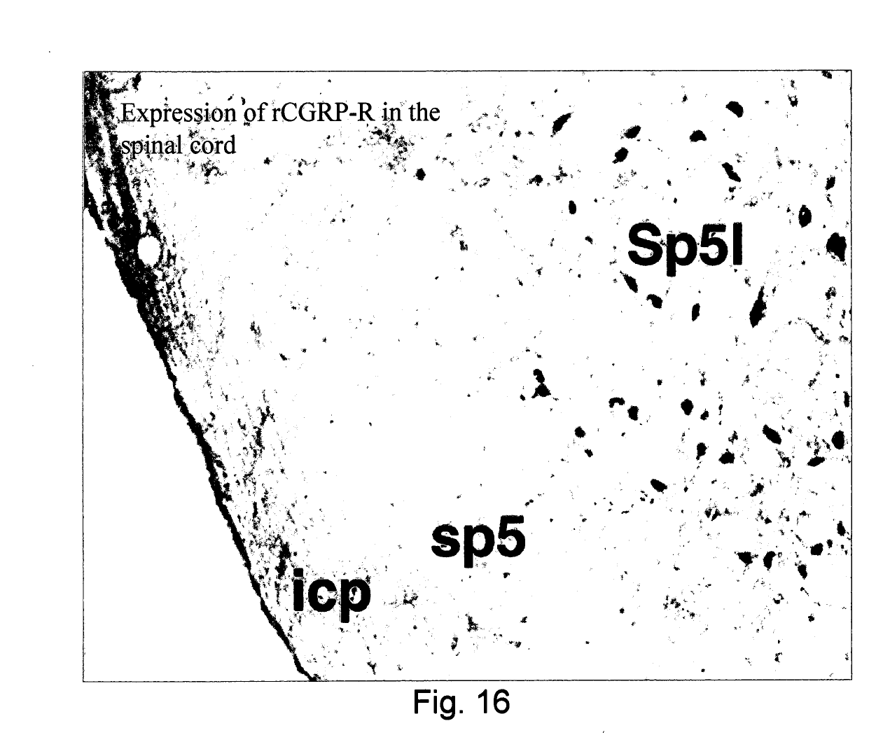

In situ hybridization studies/localizations of rat RCGRP receptors: In situ expression of the receptor of the invention was identified in select areas of spinal cord and brain, consistent with the localization of the CGRP molecule. See, FIGS. 13-16. As seen in FIGS. 13-16, in situ hybridization studies with JPr-CGRP-R cRNA probe show localization in the annotated locations.

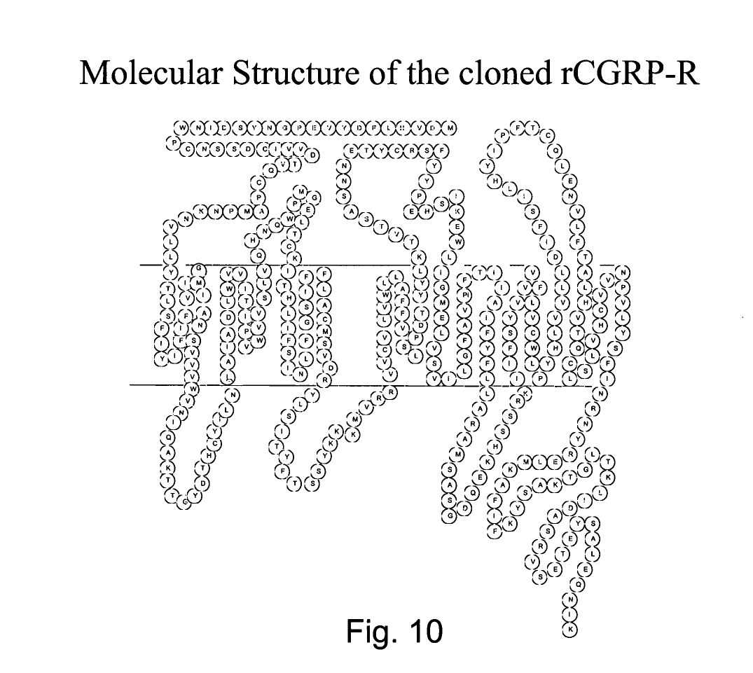

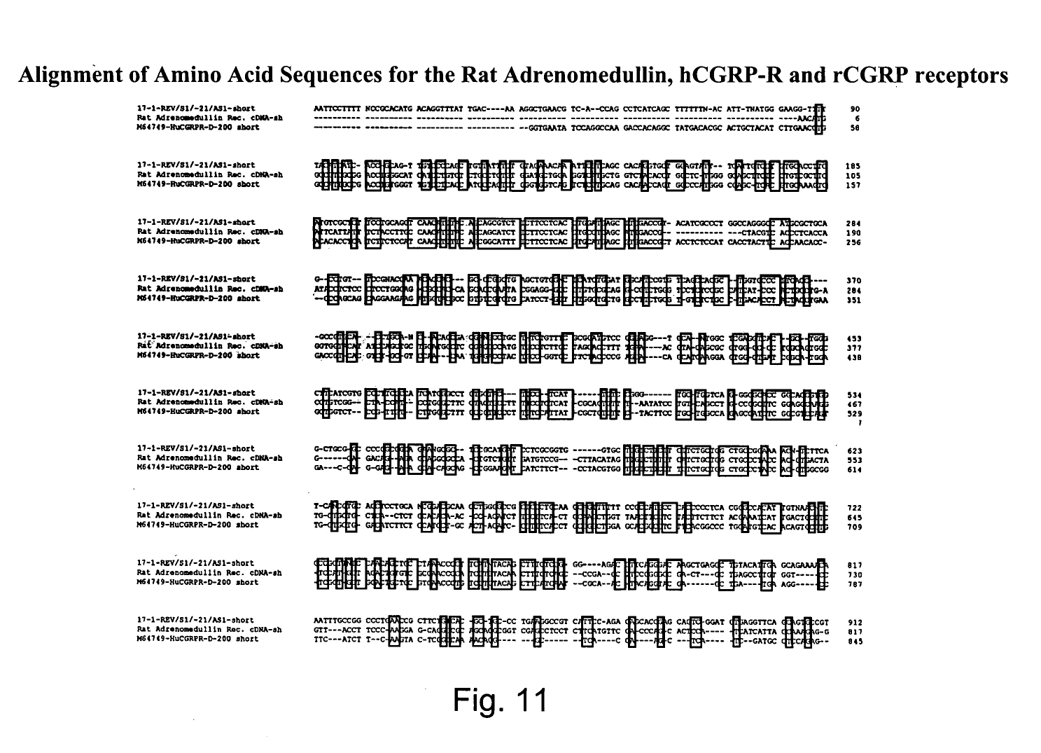

Establishment of a functional CGRP transfected cell line: A rat CGRP receptor was cloned from a cDNA library derived from a rat vas deferens cDNA library. Approximately 750,000 cDNA clones were identified and isolation of these clones was performed based on their homology with both calcitonin or adrenomedullin receptor cDNAs. Out of 750,000 cDNA clones, approximately 30 were plaque purified and sequenced using a Perkin Elmers Automated Sequencing System. NIH3T3 cells were stably transfected with the rCGRP-R, which had been sub-cloned into an pCDL-Srα vector. The cells were previously demonstrated to not express CGRP receptors and to not express RAMP proteins. Nucleotide sequencing identified two predominant species of cDNA clones that appeared to be full length. Analysis of the deduced amino acid sequence identified that the 1835 base-pair cDNA clone contained an initiation site for translation and a stop codon in frame to encode an approximately 40 kD receptor protein containing seven transmembrane domains by hydropathy plotting and having <10% sequence similarity with the CRLR receptor. See, FIGS. 10 and 11. There are 2 potential N-linked glycosylation sites (ASN-X-SER/THR) in the deduced amino acid sequence of the JPr-CGRP-R in the amino terminus. The extracellular domains contain 2 cysteine residues, which are in the amino terminus.

Reverse Transcriptase—Polymerase Chain Reaction (RT-PCR): Isolation of mRNA from all cell lines was performed using the Invitrogen Micro-FastTrack Kit. RT-PCR was performed using the Boehringer Mannheim RT-PCR kit. Briefly, fully confluent cell culture flasks (T75) generated two samples for mRNA isolation. The cells were lysed, contaminating contents removed, and the mRNA was isolated using oligo d(T) cellulose chromatography. The purified mRNA was then incubated in the presence of excess deoxynucleotides and the enzyme Reverse Transcriptase to generate cDNA. Specific sense and antisense primers spanning the intron-exon junctions were used in PCR to amplify gene sequences. Finally, amplification products were analyzed by electrophoresis in 1% agarose gels and visualized by ethidium bromide staining.

Radioligand Binding Inhibition Assay: Stably transfected NIH/3T3 cells were plated on 24-well culture dishes and incubated overnight in DMEM/10% Fetal Bovine Serum (37° Celsius). The target cell count per well was 150,000, a density high enough to ensure the cells would not detach from the substrate during the experiment. The following day, the cells were washed once in 1% PBS/BSA solution. They were then incubated for 60 minutes in a 1% DMEM/BSA solution (0.5 mL) containing 50 pM of ¹²⁵I-CGRP (DuPont, NEN, Boston, Mass.) either with or without the indicated concentrations of unlabelled peptides (3×10⁻⁸ mM, 1×10⁻⁷ mM, 3×10⁻⁷ mM to 1×10⁻⁶ mM). After the incubation, the cells were washed twice with a 1% PBS-BSA solution and solubilized with 1 mL of 0.1 N NaOH. Bound ¹²⁵I-CGRP was measured with a LKB γ-counter (Wallace Inc., Gaithesberg, Md.).

Adenylate Cyclase Stimulation Assay: Stably transfected NIH/3T3 cells were plated on 24-well culture dishes and incubated overnight with DMEM/10% Fetal Bovine Serum in the presence of Tritium (³H) Adenine at a concentration of 2 mCi/ml (37° Celsius). The target cell count per well was 150,000 cells, a density high enough to ensure the cells would not detach from the substrate during the experiment. The cells were washed with 1 ml of a nutrient deficient DMEM solution (Gibco-BRL, Gaithersburg, Md.) to remove excess radioisotope and incubated for 10 minutes (37° Celsius). The wash solution was removed and 500 μL of a DMEM solution containing 1 mg/ml BSA and 2.5 mM 3-isobutyl, 1-methylxanthine (IBMX) was added to each well. Peptide was added in 5 μL aliquots to 21 of the 24 wells with three receiving no peptide to serve as a baseline for stimulation. The remaining 21 wells were incubated at 37° Celsius with increasing concentrations of the indicated peptide and the response was measured. Following a one hour incubation, each well received 100 μL of a 2% SDS, 1 mM cAMP solution to lyse the cells. cAMP was assayed by consecutive Dowex AG-50W-X4 resin (BioRad, Richmond, Calif.) and aluminum oxide (Sigma, St. Louis, Mo.) column chromatography. Finally, 4 mL of a 10% imidazole solution eluted the contents into scintillation vials where (³H) cAMP was measured using a Beckman Liquid Scintillation Counter (Fullerton, Calif.).

Results

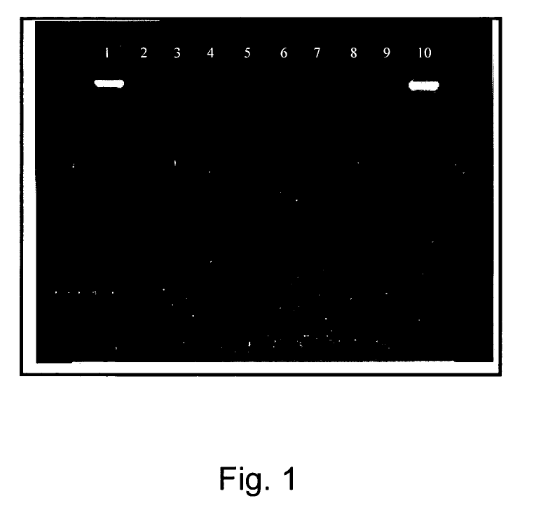

Reverse Transcriptase-Polymerase Chain Reaction (RT-PCR): RT-PCR was utilized to probe for the intracellular expression of rat JP-CGRP-R mRNA. FIG. 1 shows results of RT-PCR performed on four cell types: JP-CGRP-R transfected, Skb-CGRP-R transfected, HCT8, and SK-N-MC. HCT8 is a human colonic polyp cell line and SK-N-MC is a human neuroblastoma cell line. In FIG. 1, the Sense primer was PCDL-3176 and the antisense primer was RVD1-1KSAS. Lanes 1 and 10 in FIG. 1 show a molecular weight marker. Lane 2 shows HCT8(RT+), while Lane 3 is HCT8(RT−). Lane 4 is KD-N-MC (RT+) and Lane 5 is DK-N-MC(RT−). Lane 6 is Skb-CGRP(RT+) and Lane 7 is Skb-CGRP(RT−). Lane 8 is JP-CGRP(RT+) and Lane 9 is JP-CGRP(RT−). All cells were tested for native expression of the JP-CGRP receptor. Lane 8 displays a clear band identifying the JP-CGRP-R cDNA. Thus, JP-CGRP-R transfected cells express this gene. No bands are present in lanes 2, 4 or 6 at a distance similar to the band in lane 8. This indicates that HCT8 (lane2), SK-N-MC (lane 4), and Skb-CGRP-R transfected (lane 6) cells lines do not natively express the JP-CGRP receptor. The negative response of Skb-CGRP-R transfected cells is consistent with the significant sequence differences between the Skb-CGRP-R and JP-CGRP-R genes. The negative responses of HCT8 and SK-N-MC cells can possibly be the result of sequence differences between human and rat cell CGRP receptors (see, discussion).

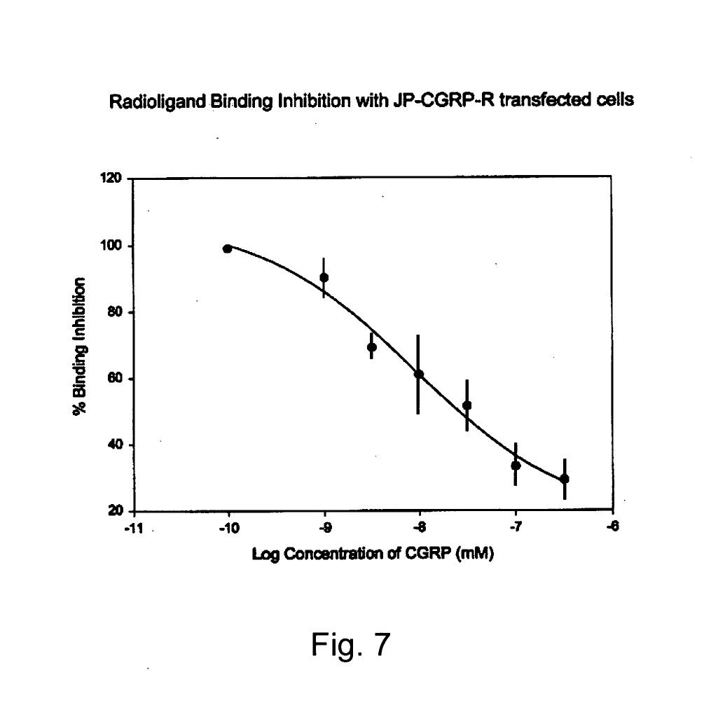

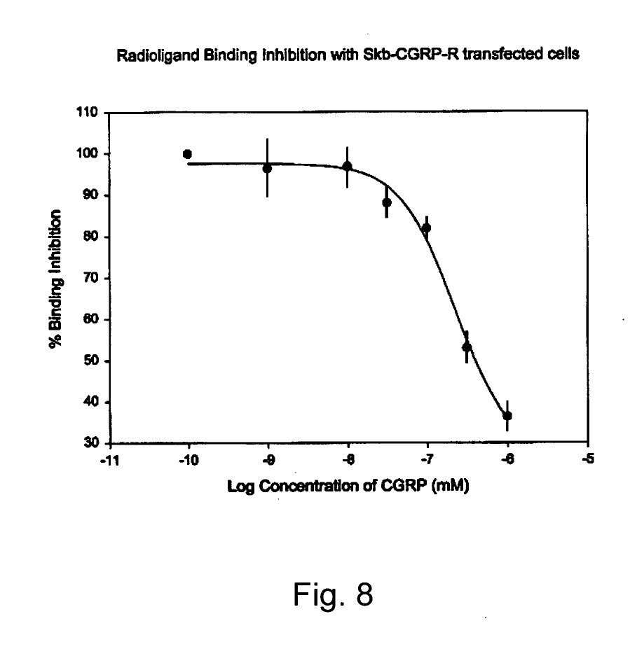

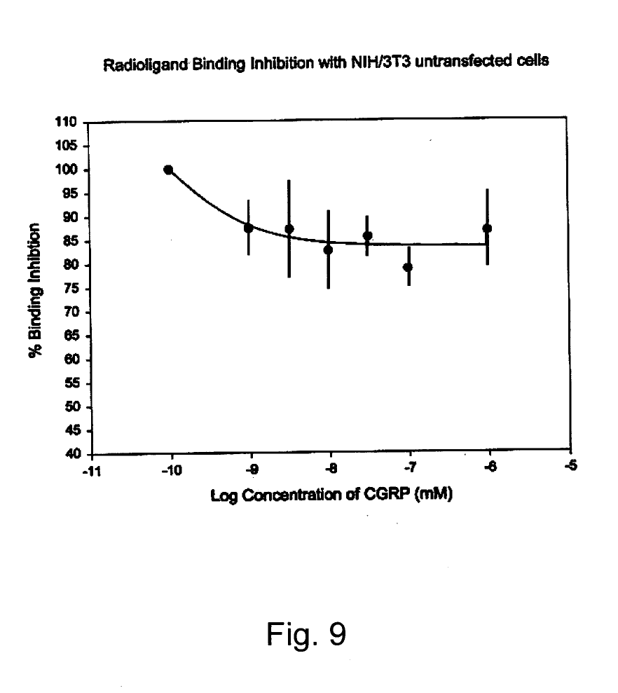

Radioligand Binding Inhibition: The Radioligand Binding Inhibition assay measures the binding potency of a peptide for its cell surface receptor. FIGS. 7, 8, and 9 show results of Binding Inhibition assays performed on rat JP-CGRP-R transfected cells, Skb-CGRP-R transfected cells, and untransfected NIH/3T3 control cells respectively. In FIG. 7, the cells were incubated in the presence of increasing concentrations of CGRP peptide. The data is shown as a percent of binding inhibition and represents the means of three experiments performed in triplicate. In FIG. 8, the cells were incubated in the presence of increasing concentrations of CGRP peptide. The data is shown as a percent of binding inhibition and represents the means of two experiments performed in triplicate. In FIG. 9, the cells were incubated similar to those in FIGS. 7 and 8 and the data is also shown as a percent of binding inhibition (from the means of three experiments performed in triplicate). As shown by these figures, CGRP dose dependently inhibits ¹²⁵I-CGRP binding for both receptor subtypes. The half-maximal inhibition (IC₅₀) value for rat JP-CGRP-R transfected cells is 3.98×10⁻⁸ mM. The IC₅₀ value for Skb-CGRP-R transfected cells is 5.01×10⁻⁷ mM. The JP-CGRP receptor's lower IC₅₀ value indicates a more potent CGRP binding than the Skb-CGRP receptor. The lack of binding inhibition in untransfected control cells suggests native CGRP receptors are not expressed on the NIH/3T3 cells. Thus, the inhibitory responses depend distinctly upon the presence of either CGRP receptor variant.

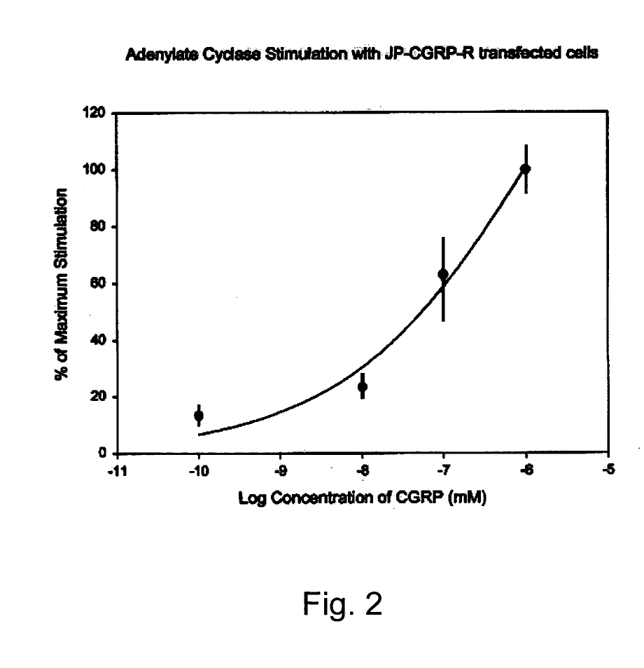

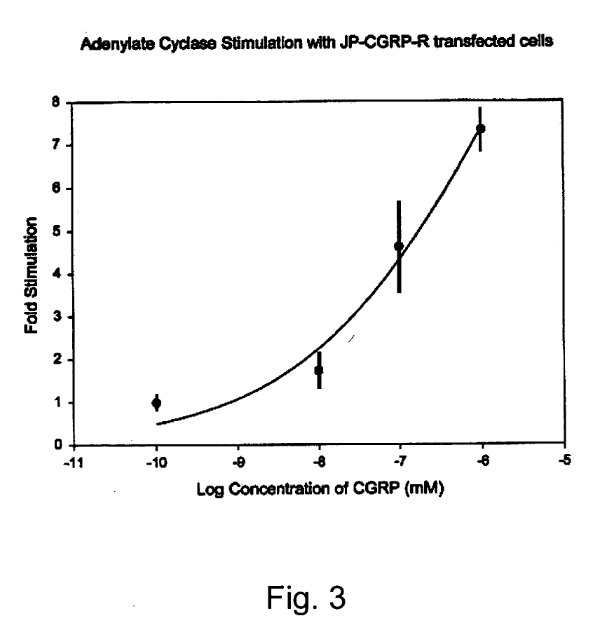

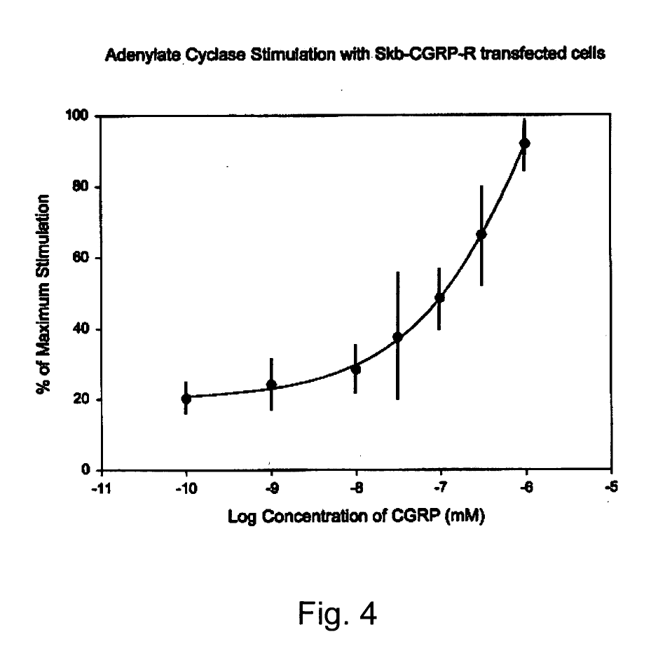

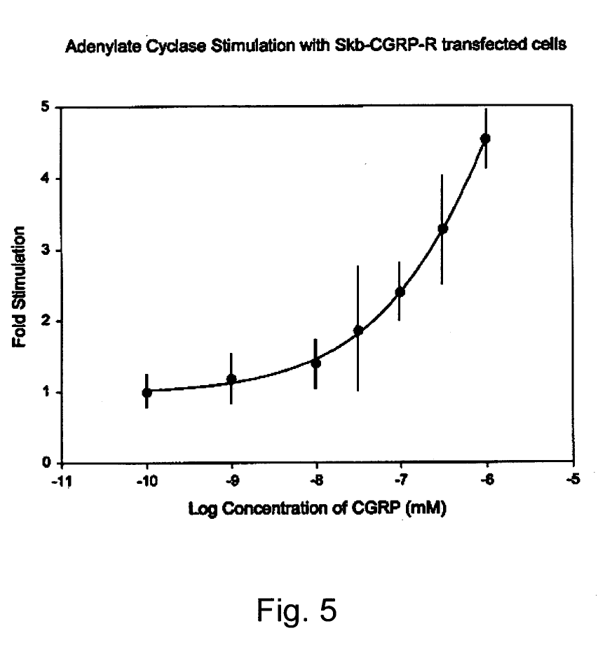

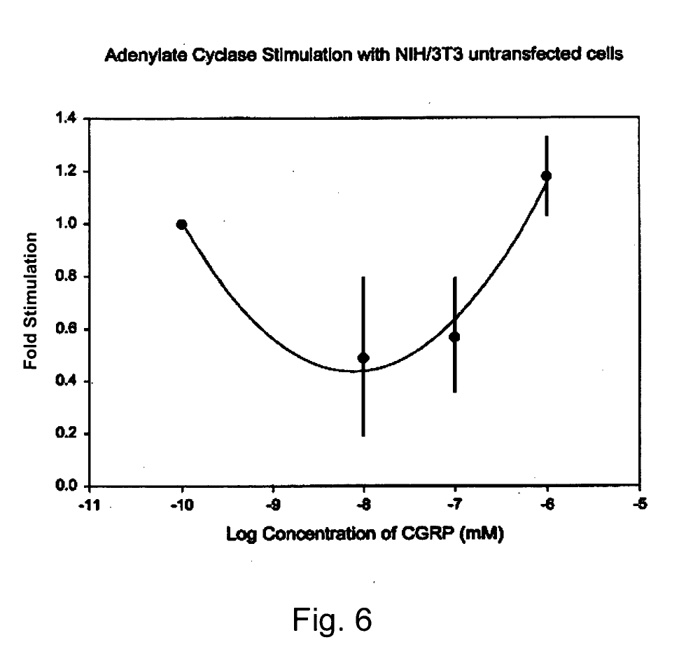

Adenylate Cyclase Stimulation: The Adenylate Cyclase Stimulation assay measures the efficacy and potency by which CGRP activates the intracellular enzyme Adenylate Cyclase. FIGS. 2 and 3 display Adenylate Cyclase Stimulation results for NIH/3T3 cells transfected with rat JP-CGRP-R cDNA. In FIG. 2, the cells were incubated in the presence of increasing concentrations of CGRP peptide, and the data is shown as a percent of maximal stimulation and represents the means of three experiments performed in triplicate. The cells in FIG. 3 were incubated similar to those in FIG. 2, but the data is shown as a fold stimulation and represents the means of three experiments performed in triplicate. FIGS. 4 and 5 show results for cells transfected with Skb-CGRP-R cDNA. In FIGS. 4 and 5 the cells were incubated in the present of increasing concentrations of CGRP peptide. The data in FIG. 4 is shown as a percent of maximal stimulation and represents the means of three experiments performed in triplicate, while the data in FIG. 5 is shown as fold stimulation and represents the means of three experiments performed in triplicate. The graphs present information in terms of “% of Maximum Stimulation” and “Fold Stimulation”. FIG. 6 displays results for untransfected NIH/3T3 control cells. In FIG. 6, the cells were incubated in the presence of increasing concentrations of CGRP peptide, and the data is shown as fold stimulation (from the means of three experiments done in triplicate). As shown by these figures, CGRP dose dependently increased cAMP concentration for both receptor subtypes. The half-maximal stimulation (EC₅₀) value for JP-CGRP-R transfected cells is 6.46×10⁻⁸ mM. The EC₅₀ value for Skb-CGRP-R transfected cells is 8.32×10⁻⁸ mM. At maximum CGRP concentrations, JP-CGRP-R and Skb-CGRP-R cells are stimulated 7.35 fold and 3.59 fold respectively, over their baseline levels. Thus, JP-CGRP-R cells have a 105% larger fold stimulation than Skb-CGRP-R cells at maximum peptide concentrations. There was no stimulation of cAMP following incubation with Amylin at doses as high as 1×10⁻⁶ mM (data not shown). Both receptors respond similarly to CGRP by activating Adenylate Cyclase. However, the JP-CGRP receptor's lower EC₅₀ value indicates a more efficacious cAMP response. Additionally, the JP-CGRP receptor's larger fold stimulation at maximum peptide concentrations indicates a more potent cAMP response. The lack of cAMP stimulation in untransfected control cells suggests native CGRP receptors are not expressed on the NIH/3T3 cells. Thus, the stimulatory responses depend distinctly upon the presence of either CGRP receptor type variant.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

| SEQ | | |
|---|---|---|
| ID | Sequence | |
| NO | Name | Sequences |
| 1 | JPr-CGRP-R | GGCAGCCGCGAAGTCACTTGGTTGCTCTCCTCAAG |
| | (na) | |
| | | TCCATGGATGTGCATCTGTTTGACTATGTGGGAAC |
| | | CTGGAACTACTCGGACATCAACTGGCCCTGTAACA |
| | | GTAGCGACTGCATCGTCGTGGACACCGTGCAGTGT |
| | | CCCGCCATGCCCAACAAGAATGTGCTGCTGTATAC |
| | | CCTCTCCTTCATCTACATTTTCATCTTCGTGATCG |
| | | GTATGATTGCCAACTCCGTGGTGGTCTGGGTGAAT |
| | | ATCCAGGCCAAGACTACAGGCTACGACACACACTG |
| | | CTACATCTTGAACCTGGCCATTGCCGATCTGTGGG |
| | | TCGTCATCACCATCCCTGTCTGGGTGGTCAGTCTC |
| | | GTGCAGCATAACCAGTGGCCCATGGGTGAGCTCAC |
| | | GTGCAAGATCACACACCTCATTTTCTCCATCAACC |
| | | TCTTTGGGAGCATCTTCTTCCTCGCATGCATGAGC |
| | | GTGGACCGCTATCTCTCCATCACCTACTTCACCAG |
| | | CACCTCCAGCTATAAGAAGAAGATGGTACGCCGTG |
| | | TTGTCTGCGTCTTGGTGTGGCTGCTGGCCTTCTTT |
| | | GTGTCCCTGCCTGACACCTACTACCTGAAGACGGT |
| | | CACATCTGCTTCCAACAACGAGACCTACTGCAGGT |
| | | CCTTCTACCCCGAGCACAGCATCAAGGAGTGGCTC |
| | | ATTGGCATGGAGCTGGTCTCCGTCATCTTGGGTTT |
| | | TGCTGTCCCCTTCACCATCATTGCTATCTTCTACT |
| | | TCCTGCTCGCCAGAGCCATGTCAGCATCCGGTGAC |
| | | CAGGAGAAACACAGCAGCCGGAAGATCATCTTCTC |
| | | CTACGTGGTGGTCTTCCTGGTGTGTTGGCTGCCGT |
| | | ACCATTTTGTGGTTCTGCTGGACATCTTCTCTATC |
| | | TTGCACTACATCCCGTTCACCTGCCAACTCGAGAA |
| | | TGTGCTCTTTACAGCGCTGCACGTCACGCAGTGCC |
| | | TGTCCCTGGTGCACTGCTGTGTCAACCCTGTGCTC |
| | | TACAGCTTCATCAACCGAAACTACAGGTACGAGCT |
| | | GATGAAGGCCTTCATCTTCAAGTACTCAGCCAAAA |
| | | CAGGACTCACCAAACTCATCGATGCCTCCAGAGTG |
| | | TCAGAGACAGAGTACTCTGCCCTGGAGCAGAACAC |
| | | CAAGTGACCGTGCTATAGGAGCATGGGGGACATGT |
| | | GCATGTTGCAAATGGGGCAGCTGGGCCCTGCGGTT |
| | | TCTTCAAGAAAAGCACTGTAGCTTTGGGTCTGGTT |
| | | GCTTGAGTGGTATGAAGAGGG |
| 2 | JPh-CGRP-R | ATGGATCTGCACCTCTTCGACTACGCCGAGCCAGG |
| | (na) | |
| | | CAACTTCTCGGACATCAGCTGGCCATGCAACAGCA |
| | | GCGACTGCATCGTGGTGGACACGGTGATGTGTCCC |
| | | AACATGCCCAACAAAAGCGTCCTGCTCTACACGCT |
| | | CTCCTTCATTTACATTTTCATCTTCGTCATCGGCA |
| | | TGATTGCCAACTCCGTGGTGGTCTGGGTGAATATC |
| | | CAGGCCAAGACCACAGGCTATGACACGCACTGCTA |
| | | CATCTTGAACCTGGCCATTGCCGACCTGTGGGTTG |
| | | TCCTCACCATCCCAGTCTGGGTGGTCAGTCTCGTG |
| | | CAGCACAACCAGTGGCCCATGGGCGAGCTCACGTG |
| | | CAAAGTCACACACCTCATCTTCTCCATCAACCTCT |
| | | TCAGCGGCATTTTCTTCCTCACGTGCATGAGCGTG |
| | | GACCGCTACCTCTCCATCACCTACTTCACCAACAC |
| | | CCCCAGCAGCAGGAAGAAGATGGTACGCCGTGTCG |
| | | TCTGCATCCTGGTGTGGCTGCTGGCCTTCTGCGTG |
| | | TCTCTGCCTGACACCTACTACCTGAAGACCGTCAC |
| | | GTCTGCGTCCAACAATGAGACCTACTGCCGGTCCT |
| | | TCTACCCCGAGCACAGCATCAAGGAGTGGCTGATC |
| | | GGCATGGAGCTGGTCTCCGTTGTCTTGGGCTTTGC |
| | | CGTTCCCTTCTCCATTATCGCTGTCTTCTACTTCC |
| | | TGCTGGCCAGAGCCATCTCGGCGTCCAGTGACCAG |
| | | GAGAAGCACAGCAGCCGGAAGATCATCTTCTCCTA |
| | | CGTGGTGGTCTTCCTTGTCTGCTGGCTGCCCTACC |
| | | ACGTGGCGGTGCTGCTGGACATCTTCTCCATCCTG |
| | | CACTACATCCCTTTCACCTGCCGGCTGGAGCACGC |
| | | CCTCTTCACGGCCCTGCATGTCACACAGTGCCTGT |
| | | CGCTGGTGCACTGCTGCGTCAACCCTGTCCTCTAC |
| | | AGCTTCATCAATCGCAACTACAGGTACGAGCTGAT |
| | | GAAGGCCTTCATCTTCAAGTACTCGGCCAAAACAG |
| | | GGCTCACCAAGCTCATCGATGCCTCCAGAGTGTCG |
| | | GAGACGGAGTACTCCGCCTTGGAGCAAAACGCCAA |
| | | G |
| 3 | JPr-CGRP-R | MDVHLFDYVEPGNYSDINWPCNSSDCIVVDTVQCP |
| | (aa) | |
| | | ANPNKNVLLYTLSFIYTFIFVIGMIANSVVVWVNI |
| | | QAKTTGYDTHCYILNLAIADLWVVITIPVWVVSLV |
| | | QHNQWPMGELTCKTTHLIFSINLFGSIFFLACMSV |
| | | DRYLSITYFTSTSSYKKKNVRRVVCVLVWLLAFFV |
| | | SLPDTYYLKTVTSASNNETYCRSFYPEHSIKEWLI |
| | | GMELVSVTLGFAVPFTHAIFYFLLARAIYSASGDQ |
| | | EKHSSRKIIFSYVVVFLVCWLPYHFVVLLDIFSIL |
| | | HYIPFTCQLENVLFTALHVTQCLSLVHCCVNPVLY |
| | | SFINRNYRYELMKAFIFKYSAKTGLTKLIDASRVS |
| | | ETEYSALEQNTK |
| 4 | JPh-CGRP-R | MDLHLFDYAEPGNFSDISWPCNSSDCIVVDTVMCP |
| | (aa) | |
| | | NNPNKSVLLYTLSFIYIFIFVIGMIAHSVVVWVNI |
| | | QAKTTGYDTHCYILNLAIADLWVVLTIPVWVVSLV |
| | | QHNQWPMGELTCKVTHLIFSINLFSGIFFLTCMSV |
| | | DRYLSITYFTNTPSSRKKMVRRVVCILVWLLAFCV |
| | | SLPDTYLKTVTSASNINETYCRSFYPEHSIKEWLI |
| | | GMELVSVVLGFAVPFSIIAVFYFLLAPAISASSDQ |
| | | EKHSSRKHFSYVVkTFLVCWLPYHVAVLLDIFSIL |
| | | HYIPFTCRLEHALFTALHVTQCLSLVHCCVNPVLY |
| | | SFINRNYRYELMKAFIFKYSAKTGLTKLIDASRVS |
| | | ETEYSALEQNAK |

What is claimed is:

1. An isolated or recombinant expression vector comprising a nucleic acid, which nucleic acid comprises a polynucleotide sequence encoding a polypeptide which binds a CGRP or CGRP-like molecule, wherein the polynucleotide sequence is selected from the group consisting of:
a) the polynucleotide sequence of SEQ ID NO:1 or SEQ ID NO: 2, or a complementary polynucleotide sequence thereof;
b) a polynucleotide sequence encoding the polypeptide sequence of SEQ ID NO:3 or SEQ ID NO:4, or a complementary polynucleotide sequence thereof;
c) a polynucleotide sequence which hybridizes under highly stringent conditions over substantially the entire length of polynucleotide sequence (a) or (b), wherein the polynucleotide sequence is unique as compared to a sequence corresponding to GenBank accession number X14048; and,
d) a polynucleotide sequence comprising a fragment of (a), (b), or (c), wherein the fragment encodes a polypeptide that binds a CGRP molecule.

2. The nucleotide of claim 1 wherein the polynucleotide sequence encodes a receptor for a CGRP molecule.

3. The receptor of claim 2, wherein the receptor is a rat CGRP receptor.

4. The receptor of claim 2, wherein the receptor is a human CGRP receptor.

5. An isolated or recombinant expression vector comprising a nucleic acid encoding a CGRP-receptor, which receptor comprises a polynucleotide sequence encoding a polypeptide, the polypeptide comprising an amino acid sequence which is substantially identical over at least 200 contiguous amino acid residues of SEQ ID NO:3 or SEQ ID NO:4.

6. The isolated or recombinant nucleic acid of claim 5, wherein binding of a CGRP molecule or CGRP-like molecule to the CRGP-receptor elicits a change in cAMP concentration.

7. The isolated or recombinant nucleic acid of claim 5, wherein the polypeptide comprises an amino acid sequence which is substantially identical over at least 300 contiguous amino acid residues of SEQ ID NO: 3 or SEQ ID NO:4; over at least 350 contiguous amino acid residues of SEQ ID NO: 3 or SEQ ID NO:4; over at least 360 contiguous amino acid residues of SEQ ID NO: 3 or SEQ ID NO:4; or over at least 361 contiguous amino acid residues of SEQ ID NO: 3 or SEQ ID NO:4.

8. The nucleic acid of claim 5, wherein the encoded polypeptide is about 362 amino acid residues in length.

9. The nucleic acid of claim 1 or 5, wherein the encoded polypeptide comprises one or more of: a leader sequence; an epitope tag sequence, a carboxy terminal epitope tag sequence, or a GFP sequence.

10. The nucleic acid of claim 1 or 5, wherein the encoded polypeptide binds a CGRP molecule or a CGRP-like molecule.

11. The nucleic acid of claim 1 or 5, wherein the nucleic acid encodes a fusion protein which comprises one or more additional nucleic acid sequences.

12. The nucleic acid of claim 1 or 5, wherein the nucleic acid encodes a polypeptide comprising a sequence having at least 91% sequence identity to SEQ ID NO:3 or to SEQ ID NO:4, wherein the polypeptide binds a CGRP molecule or a CGRP-like molecule.

13. A composition of matter comprising two or more nucleic acids of claim 1 or 5.

14. The composition of claim 13, wherein the composition comprises a library comprising at least about 2, 5, 10, 50, or more nucleic acids.

15. A composition produced by cleaving one or more nucleic acid of claim 1 or 5, wherein the cleaving comprises one or more of: mechanical cleavage, chemical cleavage, enzymatic cleavage, cleavage with a restriction endonuclease, cleavage with an RNAse, or cleavage with a DNAse.

16. A composition produced by a process comprising incubating one or more nucleic acid of claim 1 or 5 in the presence of deoxyribonucleotide triphosphates and a nucleic acid polymerase.

17. The composition of claim 16, wherein the nucleic acid polymerase is a thermostable polymerase.

18. A cell comprising one or more nucleic acid of claim 1 or 5.

19. The cell of claim 18, wherein the cell express a polypeptide encoded by the nucleic acid.

20. The cell of claim 19, wherein the expressed polypeptide comprises a CGRP receptor.

21. The receptor of claim 20, wherein the receptor is a rat receptor.

22. The receptor of claim 20, wherein the receptor is a human receptor.

23. A vector comprising the nucleic acid of claim 1 or 5.

24. The vector of claim 23, wherein the vector comprises a plasmid, a cosmid, a phage, a virus, a fragment of a virus, or an expression vector.

25. A cell transduced by the vector of claim 23.

26. An expression vector comprising the nucleic acid of claim 1 or 5.

27. An isolated or recombinant polypeptide encoded by the nucleic acid of claim 1 or 5.

28. The isolated or recombinant polypeptide of claim 27, wherein the polypeptide comprises an amino acid sequence of SEQ ID NO:3 or SEQ ID NO:4.

29. The isolated or recombinant polypeptide of claim 27, wherein the encoded polypeptide binds a CGRP molecule or a CGRP-like molecule.

30. The isolated or recombinant polypeptide of claim 29, wherein the CGRP molecule or CGRP-like molecule bound to the polypeptide elicits a change in cAMP concentration.

31. A composition comprising the isolated or recombinant polypeptide of claim 27, wherein the composition comprises the polypeptide bound to a CGRP molecule or to a CGRP-like molecule.

32. An isolated or recombinant polypeptide, which polypeptide binds a CGRP molecule or a CGRP-like molecule, and which polypeptide is selected from the group consisting of:
a) a polypeptide encoded by a polynucleotide sequence of SEQ ID NO:1 or SEQ ID NO:2;
b) a polypeptide encoded by SEQ ID NO:3 or SEQ ID NO:4;
c) a polypeptide encoded by a polynucleotide sequence which hybridizes under highly stringent conditions over substantially the entire length of a polynucleotide sequence encoding (a) or (b), wherein the polynucleotide sequence does not comprise a sequence corresponding to GenBank accession number X14084; and,
d) a polypeptide comprising a sequence having at least 91% sequence identity to SEQ ID NO:3 or to SEQ ID NO:4, wherein the polypeptide binds a CGRP molecule or a CGRP-like molecule.

33. A composition comprising the isolated or recombinant polypeptide of claim 32, wherein the composition comprises the polypeptide bound to a CGRP molecule or to a CGRP-like molecule.

34. The isolated or recombinant polypeptide of claim 33, wherein the CRGP molecule or the CGRP-like molecule bound to the polypeptide elicits a change in cAMP concentration.

35. The polypeptide of claim 32, wherein the encoded polypeptide binds a CGRP molecule or a CGRP-like molecule

36. The isolated or recombinant polypeptide of claim 32, wherein the polypeptide comprises a CGRP receptor.

37. The polypeptide of claim 35, wherein the CGRP molecule or CGRP-like molecule bound to the polypeptide elicits a change in cAMP concentration.

38. The isolated or recombinant polypeptide of claim 32, comprising a polypeptide encoded by a polynucleotide sequence, which polynucleotide sequence hybridizes under highly stringent conditions over substantially the entire length of: a polynucleotide sequence of SEQ ID NO:1 or SEQ ID NO:2, or a complementary sequence thereof, or a polynucleotide sequence encoding a polypeptide sequence of SEQ ID NO:3 or SEQ ID NO:4.

39. The isolated or recombinant polypeptide of claim 32, wherein the polypeptide comprises at least 92% sequence identity to SEQ ID NO:3 or SEQ ID NO:4; at least 93% sequence identity to SEQ ID NO:3 or SEQ ID NO:4; at least 94% sequence identity to SEQ ID NO:3 or SEQ ID NO:4; at least 95% sequence identity to SEQ ID NO:3 or SEQ ID NO:4; at least 96% sequence identity to SEQ ID NO:3 or SEQ ID NO:4; at least 97% sequence identity to SEQ ID NO:3 or SEQ ID NO:4; at least 98% sequence identity to SEQ ID NO:3 or SEQ ID NO:4; or at least 99% sequence identity or more to SEQ ID NO:3 or SEQ ID NO:4.

40. The isolated or recombinant polypeptide of claim 32, wherein the polypeptide comprises an amino acid sequence which is substantially identical over at least 200 contiguous amino acid residues of SEQ ID NO: 3 or SEQ ID NO:4; over at least 300 contiguous amino acid residues of SEQ ID NO: 3 or SEQ ID NO:4; over at least 350 contiguous amino acid residues of SEQ ID NO: 3 or SEQ ID NO:4; over at least 360 contiguous amino acid residues of SEQ ID NO: 3 or SEQ ID NO:4; or over at least 361 contiguous amino acid residues of SEQ ID NO: 3 or SEQ ID NO:4.

41. The isolated or recombinant polypeptide of claim 32, which is substantially identical over 362 amino acids of the encoded polypeptide.

42. An isolated or recombinant polypeptide comprising an amino acid sequence of SEQ ID NO:3 or SEQ ID NO:4, wherein the polypeptide comprises a receptor capable of binding a CGRP molecule or a CGRP-like molecule, wherein such binding elicits a change in cAMP concentration.

43. The isolated or recombinant polypeptide of claim 27, 32, or 42, comprising one or more of: a leader sequence, a precursor polypeptide, a secretion signal, a localization signal, an epitope tag, an E-tag, or a His epitope tag.

44. A method of producing a polypeptide, the method comprising:
introducing a nucleic acid of claim 1 or 5 into a population of cells, which nucleic acid is operably linked to a regulatory sequence capable of directing expression of the encoded polypeptide in at least a subset of cells or progeny thereof; and propagating the cells, thereby producing the polypeptide.

45. The method of claim 44, further comprising isolating the polypeptide from the cells or from the culture medium.

46. The method of claim 44 or 45, wherein the culturing is performed in a bulk fermentation vessel.

47. The method of claim 44 or 45, wherein the cells are selected from the group consisting of: bacterial cells, eukaryotic cells, fungal cells, yeast cells, plant cells, insect cells, and mammalian cells.

48. The method of claim 47, wherein the mammalian cells comprise fertilized oocytes, embryonic stem cells, or pluripotent stem cells, further comprising regenerating a transgenic mammal expressing the polypeptide, and recovering the polypeptide from the transgenic mammal or from a by-product of the transgenic mammal.

49. The method of claim 47, wherein the mammalian cells comprise fertilized oocytes, embryonic stem cells, or pluripotent stem cells, further comprising regenerating a transgenic mammal expressing the polypeptide, and wherein the polypeptide is overexpressed or comprises a knockout polypeptide.

50. The method of claim 49, wherein the overexpressed polypeptide or the knockout polypeptide is localized to a particular tissue or cell type in the transgenic animal.

51. A method of producing an isolated or recombinant polypeptide, the method comprising:
(a) introducing into a population of cells a recombinant expression vector comprising a nucleic acid of claim 1 or 5;
(b) administering the expression vector into a mammal; and,
(c) isolating the polypeptide from the mammal or from a byproduct of the mammal.

52. An isolated or recombinant polypeptide comprising a receptor capable of binding a CGRP molecule or a CGRP-like molecule, wherein such binding elicits a change in cAMP concentration, which polypeptide is specifically bound by a polyclonal antisera raised against at least one antigen, said at least one antigen comprising SEQ ID NO:3, or a fragment thereof, or of SEQ ID NO:4 or a fragment thereof, wherein the antisera is subtracted with a polypeptide encoded by a nucleic acid corresponding to GenBank accession number X14084.

53. An antibody or antisera produced by administering the isolated or recombinant polypeptide of claim 32 to a mammal, which antibody or antisera specifically binds at least one antigen, said at least one antigen comprising a polypeptide comprising the amino acid sequence of SEQ ID NO:3 or SEQ ID NO:4, which antibody or antisera does not specifically bind to a peptide encoded by a nucleic acid corresponding to GenBank accession number X14084.

54. An isolated or recombinant expression vector comprising a nucleic acid which comprises a unique subsequence in a nucleic acid represented by SEQ ID NO:1 or SEQ ID NO:2, wherein the unique subsequence is unique as compared to a nucleic acid sequence of GenBank accession number X14084.

55. An isolated or recombinant polypeptide which comprises a unique subsequence in a polypeptide represented by SEQ ID NO:3 or SEQ ID NO:4, wherein the unique subsequence is unique as compared to a polypeptide sequence corresponding to a wild-type CGRP receptor represented by GenBank accession number X14084.

56. A composition comprising the isolated or recombinant polypeptide of claim 55, wherein the composition comprises the polypeptide bound to a CGRP molecule or to a CGRP-like molecule.

57. A method of modulating the activity of a CGRP receptor, wherein the receptor comprises a polypeptide sequence of claim 32, the method comprising:
binding a CGRP molecule or a CGRP-receptor agonist or a CGRP-receptor antagonist to the CGRP receptor.

58. The method of claim 57, wherein the receptor comprises a polypeptide sequence that comprises at least 92% or more identity to SEQ ID NO:3 or SEQ ID NO:4; at least 93% or more identity to SEQ ID NO:3 or SEQ ID NO:4; at least 94% or more identity to SEQ ID NO:3 or SEQ ID NO:4; at least 95% or more identity to SEQ ID NO:3 or SEQ ID NO:4; at least 96% or more identity to SEQ ID NO:3 or SEQ ID NO:4; at least 97% or more identity to SEQ ID NO:3 or SEQ ID NO:4; at least 98% or more identity to SEQ ID NO:3 or SEQ ID NO:4; or at least 99% or more identity to SEQ ID NO:3 or SEQ ID NO:4.

59. The method of claim 57, wherein the CGRP receptor comprises a human CGRP receptor.

60. The method of claim 57, wherein the CGRP receptor comprises a rat CGRP receptor

61. A method of performing high throughput screening of CGRP-receptor modulating compounds, the method comprising: interacting a putative CGRP-receptor modulating compound with a CGRP receptor, wherein the receptor comprises a polypeptide sequence comprising at least 91% or more identity to SEQ ID NO:3 or SEQ ID NO:4; at least 92% or more identity to SEQ ID NO:3 or SEQ ID NO:4; comprises at least 93% or more identity to SEQ ID NO:3 or SEQ ID NO:4; at least 94% or more identity to SEQ ID NO:3 or SEQ ID NO:4; at least 95% or more identity to SEQ ID NO:3 or SEQ ID NO:4; at least 96% or more identity to SEQ ID NO:3 or SEQ ID NO:4; comprises at least 97% or more identity to SEQ ID NO:3 or SEQ ID NO:4; at least 98% or more identity to SEQ ID NO:3 or SEQ ID NO:4; or at least 99% or more identity to SEQ ID NO:3 or SEQ ID NO:4.
